(19) United States
(12) Patent Application Publication
(10) Pub. No.: US 2024/0009092 A1
(43) Pub. Date: Jan. 11, 2024

(54) COSMETIC COMPOSITION AND COSMETIC QUASI-DRUG INCLUDING LIPID PARTICLES CONTAINING PHOSPHOLIPID

(71) Applicant: NIPPON SHOKUBAI CO., LTD., Osaka (JP)

(72) Inventor: Morihiro KITAMURA, Osaka (JP)

(73) Assignee: NIPPON SHOKUBAI CO., LTD., Osaka (JP)

(21) Appl. No.: 18/035,194

(22) PCT Filed: Nov. 11, 2021
(86) PCT No.: PCT/JP2021/041637

§ 371 Date: May 3, 2023

(30) Foreign Application Priority Data
Nov. 13, 2020 (JP) 2020-189158

(51) Int. Cl. A61K 8/14; A61Q 19/00; A61K 8/34

(52) U.S. Cl. CPC A61K 8/14; A61Q 19/00; A61K 8/345; A61K 2800/10

(57) ABSTRACT

The purpose of the present invention is to provide a novel cosmetic composition or cosmetic quasi-drug which has excellent storage stability. The cosmetic composition or cosmetic quasi-drug comprising: lipid particles containing at least a phospholipid; a polyhydric alcohol; and water, wherein a content of a trihydric or higher alcohol in the polyhydric alcohol is 0 to 20% by mass relative to 100% by mass of the polyhydric alcohol.

### TECHNICAL FIELD

The present invention relates to a cosmetic composition or cosmetic quasi-drug including lipid particles containing a phospholipid.

More specifically, the present invention relates to a cosmetic composition or cosmetic quasi-drug including lipid particles containing a phospholipid, the cosmetic composition or cosmetic quasi-drug being used in skin cosmetics, skin external preparations, and the like.

### BACKGROUND ART

Phospholipids have been conventionally used, as raw materials for lipid particles, in various cosmetic products, quasi-drugs, and pharmaceutical drugs applications. Phospholipids are especially used as a main raw material for a liposome, which has a bimolecular membrane structure composed of lipid.

For example, Patent Literature 1 discloses a liposome characterized by containing a phospholipid (component A), a sterol (component B), a polyoxyethylene sterol ether (component C) having an HLB value of from 12.0 to 16.0, a polyhydric alcohol (component D), and water (component E), and discloses also that a stable liposome with a particle size of 50 nm or less can be prepared.

For example, Patent Literature 2 discloses a stable unilamellar liposomal suspension comprising: a liposome preparation suspended in an external phase composition, the liposome preparation comprising a plurality of unilamellar liposomal particles having a mean particle size between about 50 nm to about 290 nm, the liposome preparation formed from an aqueous liposomal solution comprised of an oil-soluble composition and a water-soluble composition, the oil-soluble composition at a concentration between about 5% to about 33% by weight of the liposomal solution, the water-soluble composition at a concentration between about 67% to about 95% by weight of the liposomal solution, wherein the oil-soluble composition comprises a coupling agent, at least one phospholipid, at least one rigidity enhancer, and an antioxidant; wherein the external phase composition is at a concentration between about 30% to about 75% by weight of the liposomal suspension, the external phase composition comprising a density between about 0.95 g/cc and about 1.25 g/cc and a viscosity between about 2.5 cP and about 40,000 cP at a shear rate of 10 sec⁻¹ at 21° C.; and wherein the liposomal suspension has a refractive index between about 1.30 and about 1.45, and retains its stability in neat form at a temperature of between about 4° C. to about 50° C. for a period of at least 30 days or at 21° C. for a period of at least 180 days.

For example, Patent Literature 3 discloses a cosmetic including a phospholipid vesicle composition including components (A) to (C): (A) diisostearyl malate having a 25° C. viscosity of 100 to 300 mPa-s; (B) phospholipid; and (C) polyhydric alcohol.

### RELATED ART DOCUMENTS

### Patent Documents

Patent Document 1: Japanese Unexamined Patent Application Publication No. 2007-291035
Patent Document 2: International Application Japanese-Phase Publication No. 2012-504620
Patent Document 3: Japanese Unexamined Patent Application Publication No. 2020-2097

### SUMMARY OF THE INVENTION

### Problems to be Solved by the Invention

Liposomes containing a phospholipid have been reported as described in the above Literatures. However, in order to suitably use those for a cosmetic composition or the like, there has been a demand for a cosmetic composition or the like containing lipid particles that has good storage stability while taking into consideration the properties required for cosmetics, such as use feeling.

Accordingly, an object of the present invention is to provide a cosmetic composition or the like containing lipid particles that has good storage stability and can be suitably used as a cosmetic composition or the like.

### Solutions to the Problems

The inventor made various studies to attain the above-mentioned object, and as a result, arrived at the present invention.

The cosmetic composition or cosmetic quasi-drug of the present disclosure includes lipid particles containing at least a phospholipid, a polyhydric alcohol, and water, wherein the content of a trihydric or higher alcohol in the polyhydric alcohol is 0 to 20% by mass relative to 100% by mass of the polyhydric alcohol.

### Effects of the Invention

The cosmetic composition or cosmetic quasi-drug of the present disclosure is excellent in use feeling and long-term storage stability because the amount of trihydric or higher alcohol used is small. More preferably, the cosmetic composition or cosmetic quasi-drug of the present disclosure is excellent in skin permeability, retention in the skin, and skin barrier properties. Therefore, the cosmetic composition or cosmetic quasi-drug of the present disclosure can be suitably used, for example, as an additive for a cosmetic product and a quasi-drug, and a skin external preparation.

### BRIEF DESCRIPTION OF THE DRAWINGS

FIG. 1 is an image of a lipid particle of Production Example 2 observed with a transmission electron microscope.

FIG. 2 is a schematic view of a vertical Franz diffusion cell.

FIG. 3 is a fluorescence observation image of a cryosection 120 minutes after application of a solution of Production Example 9.

FIG. 4 is a fluorescence observation image of a cryosection 120 minutes after application of a Tris buffer solution.

FIG. 5 is a graph showing a cumulative permeation amount of fluorescent dye in a permeability test of a three-dimensional skin model coated with a solution of Production Example 9.

FIG. 6 is a graph showing a cumulative permeation amount of caffeine in a receiver solution in a skin barrier test of a three-dimensional skin model using a solution of Production Example 9 and a Tris buffer solution.

FIG. 7 is an image of a lipid particle of Production Example 10 observed with a transmission electron microscope.

FIG. 8 is a graph showing a cumulative permeation amount of caffeine using a three-dimensional skin model using a solution of Production Example 30.

### MODE FOR CARRYING OUT THE INVENTION

Hereinafter, the present disclosure will be described in detail.

Any combination of two or more of the following preferred embodiments of the present disclosure is also a preferred embodiment of the present disclosure.

### [Cosmetic Composition or Cosmetic Quasi-Drug of the Present Disclosure]

The cosmetic composition or cosmetic quasi-drug of the present disclosure (hereinafter also referred to as “cosmetic composition or the like of the present disclosure”) contains, as essential components, lipid particles containing at least a phospholipid; a polyhydric alcohol; and water. The quasi-drug in the present disclosure is a quasi-drug stipulated in the Pharmaceutical Affairs Act and designated by the Minister of Health, Labour and Welfare as stipulated in Article 2, Paragraph 2 of the Pharmaceutical Affairs Act. Specifically, the quasi-drug refers to items that are used for the purpose of diagnosing, treating, ameliorating, alleviating, managing, or preventing human or animal diseases, and that are less effective than pharmaceutical drugs. For example, according to the Pharmaceutical Affairs Act, the quasi-drug is a product other than a product used for the purpose of pharmaceutical drugs, and including products used to treat or prevent diseases in humans or animals, and having little or no direct action on the human body. Components constituting quasi-drugs are listed in the Japanese Pharmacopoeia, the Specifications and Standards for Food Additives, the Japanese Industrial Standards, and the Japanese Standards of Quasi-drug Ingredients.

### <Lipid Particles Containing at Least a Phospholipid>

In the present disclosure, lipid particles containing at least a phospholipid means particles composed of lipid containing a phospholipid.

### <Phospholipid>

In the present disclosure, a phospholipid is, for example, a compound having a structure that a fatty acid and phosphoric acid are bonded to glycerin or sphingosine as the central skeleton, and alcohol further is bonded to phosphoric acid via an ester bond. The phospholipid includes natural phospholipids, synthetic phospholipids, and hydrogenated phospholipids obtained by saturating unsaturated carbon chains of naturally occurring phospholipids with hydrogen. As the fatty acid, for example, a saturated fatty acid or unsaturated fatty acid having 5 to 30 carbon atoms is preferable, and a linear saturated fatty acid or linear unsaturated carbon chain having 10 to 25 carbon atoms is preferable. Specific examples of the phospholipid include natural phospholipids such as phosphatidylcholine, phosphatidylethanolamine, phosphatidylserine, phosphatidylinositol, phosphatidylglycerol, egg-yolk lecithin, and soybean lecithin; synthetic phospholipids such as dilauroyl phosphatidylcholine, dimyristoyl phosphatidylcholine, dipalmitoyl phosphatidylcholine, distearoyl phosphatidylcholine, dioleoyl phosphatidylcholine, and palmitoyl-oleoyl phosphatidylcholine; and hydrogenated phospholipids such as hydrogenated soybean lecithin, hydrogenated egg-yolk lecithin, hydrogenated phosphatidylcholine, and hydrogenated phosphatidylserine. The phospholipid of the present disclosure is preferably glycerophospholipid in which two fatty acids and one optionally esterified phosphoric acid are bonded to glycerin, more preferably a phosphatidylcholine in which fatty acids are bonded to two of the three hydroxyl groups of glycerin, phosphoric acid is bonded to one of the three hydroxyl groups of glycerin, and choline is bonded to phosphoric acid as an alcohol. The phospholipid of the present disclosure is still more preferably hydrogenated phosphatidylcholine in which an unsaturated carbon chain of fatty acid is saturated with hydrogen. The raw material for the phospholipid is, but not particularly limited to, preferably egg-yolk phospholipid and soybean phospholipid, more preferably soybean phospholipid, and still more preferably hydrogenated soybean phospholipid. These phospholipids can be used singly or in combination of two or more.

The phospholipid of the present disclosure means a phospholipid having two acyl groups. As used herein, a lysophospholipid is not included in the phospholipid, and both are distinguished from each other to be used.

### <Lipid Particles>

In the present disclosure, only the phospholipid described above may be included as a lipid, but lipids other than the phospholipid described above may also be included. Lipids other than the phospholipid include, for example, a simple lipid. A simple lipid is composed of only three elements C, H, and O, and is an ester-type lipid formed by bonding a fatty acid and an alcohol. An alcohol that bonds to a fatty acid includes long-chain alcohols, polyhydric alcohols such as glycerol, and sterols such as cholesterol and phytosterol. Lipids other than the phospholipid include, for example, a complex lipid. A complex lipid contains three elements C, H, and O, as well as N as a base, P as a phosphate, and the like. Complex lipids other than the phospholipid include, for example, sphingolipid and glycolipid. Lipids other than the phospholipid include also, for example, unsaponifiable lipids such as alkanes, pigments, and fat-soluble vitamins. The lipids other than the phospholipid can be used singly or in combination of two or more.

The lipid particles of the present disclosure may contain a sterol and a lysophospholipid as lipids other than the phospholipid. It is preferable to use a sterol from the viewpoint of stabilization of lipid particles, and it is preferable to use a lysophospholipid from the viewpoint of particle size reduction of lipid particles.

The sterol of the present disclosure include cholesterol, which is a compound widely distributed in bodies of mammals, including humans, and fishes, and phytosterol contained in plants. It is preferable to use cholesterol.

The cholesterol of the present disclosure can be industrially obtained by extracting and purifying mainly from wool fat. In addition, cholesterol can be obtained from cattle or pig brain and spinal cord, fish oil, squid liver oil, and the like, but it is preferable to use cholesterol obtained from wool fat. In the present invention, cholesterol derived from any may be used, and one or two or more kinds of cholesterol may be used as necessary. The cholesterol of the present disclosure may also be ester-modified.

The lysophospholipid of the present disclosure means a phospholipid having one acyl group. As used herein, the lysophospholipid is not included in the phospholipid, and both are distinguished from each other to be used.

The lysophospholipid of the present disclosure may be, for example, formed by removing one fatty acid molecule bonded to the 1- or 2-position of glycerol of a glycerophospholipid by hydrolysis.

The lysophospholipid of the present disclosure has different chemical properties from those of a phospholipid with a double-chain fatty acid. Specific examples of the lysophospholipid include lysophosphatidylcholine, lysophosphatidylethanolamine, lysophosphatidylserine, lysophosphatidylinositol, lysophosphatidic acid, lysophosphatidylglycerol, and a mixture of two or more of those. Among these lysophospholipids, lysophosphatidylcholine is preferred from the viewpoint of transparently dispersing oil in water. Further preferred is a hydrogenated lysophosphatidylcholine in which the unsaturated carbon chain of the fatty acid is saturated with hydrogen.

A raw material for the lysophospholipid is not particularly limited, and examples thereof include soybean lysophospholipid, hydrogenated soybean lysophospholipid, egg-yolk lysophospholipid, and hydrogenated egg-yolk lysophospholipid. Soybean lysophospholipid is preferable, and hydrogenated soybean lysophospholipid is more preferable. These lysophospholipids can be used singly or in combination of two or more as necessary.

The lysophospholipid of the present disclosure may have a structure represented by the following general formula (1).

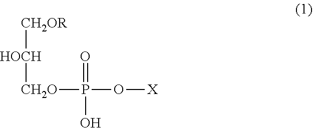

wherein R represents a saturated or unsaturated aliphatic acyl group having 10 to 30 carbon atoms, and X represents a hydrogen atom or a polar group.

R in the general formula (1) represents a saturated or unsaturated aliphatic acyl group having 10 to 30 carbon atoms. The saturated aliphatic acyl group having 10 to 30 carbon atoms is not particularly limited, and examples thereof include lauroyl group, myristoyl group, palmitoyl group, stearoyl group, eicosanoyl group, heneicosanoyl group, docosanoyl group, tricosanoyl group, tetracosanoyl group, pentacosanoyl group, hexacosanoyl group, heptacosanoyl group, octacosanoyl group, nonacosanoyl group, and triacontanoyl group. The unsaturated aliphatic acyl group having 10 to 30 carbon atoms includes monounsaturated and polyunsaturated ones, and examples thereof include docosamonoenoyl group, docosadienoyl group, docosatrienoyl group, docosatetraenoyl group, docosapentaenoyl group, docosahexaenoyl group, tricosamonoenoyl group, tricosadienoyl group, oleoyl group, linoleoyl group, linolenoyl group, and arachidonoyl group. The acyl group is preferably a saturated or unsaturated aliphatic acyl group having 14 to 20 carbon atoms, more preferably palmitoyl group, stearoyl group, linoleoyl group, oleoyl group, linolenoyl group, and the like.

X in the general formula (1) represents a hydrogen atom or a polar group. Examples of the polar group include a residue obtained by removing an OH group bonded to the carbon skeleton of a hydroxyl group-containing compound such as choline, ethanolamine, inositol, serine, glycerol, and ethanol (preferably, an aliphatic hydrocarbon having 2 to 10 carbon atoms containing a hydroxyl group and optionally at least one selected from an amino group, an alkylamino group and a carboxylic acid group). The above X is preferably a hydrogen atom or a residue of ethanolamine, choline, or glycerol, more preferably a residue of glycerol.

In the above-mentioned lipid particles, the content of the phospholipid relative to 100% by mass of the total of the phospholipid and lipids other than the phospholipid is preferably 60% by mass or more, more preferably 70% by mass or more, and still more preferably 90% by mass or more. In the phospholipid of the present disclosure, the content of the phosphatidylcholine relative to 100% by mass of the total of the phosphatidylcholine and phospholipids other than the phosphatidylcholine is preferably 60% by mass or more, more preferably 65% by mass or more, and particularly preferably 70% by mass or more. When these contents are within the above ranges, the storage stability of the cosmetic composition or the like of the present disclosure tends to be further improved. A lipid particle containing a phospholipid is amphiphilic due to the presence of a fatty acid moiety as a hydrophobic moiety and a phosphate moiety as a hydrophilic moiety in the structure, and has a spherical structure with the hydrophilic moiety facing outward in an aqueous solution. The lipid particles containing a phospholipid has such a spherical structure and therefore has a particle shape, and the particle size and the like can be measured as a particle.

The content of the phospholipid in 100 parts by mass of the lipid particles of the present disclosure is preferably 5 parts by mass or more, more preferably 30 parts by mass or more, and still more preferably 45 parts by mass or more, and is preferably 95 parts by mass or less, more preferably 90 parts by mass or less, and still more preferably 80 parts by mass or less, from the viewpoint of the stability, transdermal permeability, and skin barrier properties of the cosmetic composition or the like.

The content of the cholesterol in 100 parts by mass of the lipid particles of the present disclosure is preferably 1 part by mass or more, more preferably 5 parts by mass or more, and still more preferably 8 parts by mass or more, and is preferably 35 parts by mass or less, more preferably 30 parts by mass or less, and still more preferably 25 parts by mass or less, from the viewpoint of the stability of the cosmetic composition or the like.

The content of the lysophospholipid in 100 parts by mass of the lipid particles of the present disclosure is preferably 0.01 parts by mass or more, more preferably 0.1 parts by mass or more, and still more preferably 1 part by mass or more, and is preferably 30 parts by mass or less, more preferably 28 parts by mass or less, and still more preferably 25 parts by mass or less, from the viewpoint of the stability of the cosmetic composition or the like.

The lipid particles of the present disclosure may contain a ceramide. A ceramide is a kind of sphingolipid and can form lipid particles.

The ceramide of the present disclosure is not limited as long as it can be used in cosmetics and the like, but can be defined as the following. That is, the ceramide includes a series of ceramides expressed as a nonionic amphiphilic substance having one or more long linear (for example, 10 or more carbon atoms) and/or branched alkyl or alkenyl groups and further having at least two or more hydroxyl groups and one or more amide groups (and/or amino groups) in the molecule, or a derivative in which a phosphatidylcholine residue or a sugar residue is bonded to a hydroxyl group of the nonionic amphiphilic substance. Examples of the ceramide include natural ceramides such as ceramide EOS, ceramide NS, ceramide NP, ceramide NG, ceramide EOH, ceramide AS, ceramide AG, ceramide AP, ceramide AH, ceramide NH, ceramide EOP, ceramide NDS, ceramide ADS, ceramide EODS, and ceramide 3B, which are sphingosine, phytosphingosine, and their long-chain fatty acid amides; sphingophospholipids such as sphingomyelin and phytosphingomyelin, which are phospholipid derivatives of sphingosine and phytosphingosine; and glycosides thereof, namely, sphingoglycolipid and phytosphingoglycolipid such as cerebroside and ganglioside. These can be used singly or in combination of two or more. In the present invention, although the ceramides also include synthetic ceramides, pseudoceramides, and the like, natural ceramides are preferable among the ceramides in light of the superiority of the technology of the present invention. Ceramide NS, ceramide NG, ceramide NP, ceramide 3B, and ceramide AP are preferable for obtaining particularly excellent effects of improving skin problems and moisture retention.

The content of the ceramide in 100 parts by mass of the lipid particles of the present disclosure is preferably 0.1 parts by mass or more, more preferably 0.5 parts by mass or more, and still more preferably 1 part by mass or more from the viewpoint of the skin barrier properties, and is preferably 50 parts by mass or less, more preferably 40 parts by mass or less, and still more preferably 30 parts by mass or less from the viewpoint of the stability of the cosmetic composition or the like.

When the lipid particles contain a lysophospholipid, the mass ratio of the phospholipid and the lysophospholipid (mass of phospholipid/mass of lysophospholipid) in the lipid particles of the present disclosure is preferably 0.1/1 to 40/1, more preferably 1/1 to 35/1, and still more preferably 3/1 to 30/1 from the viewpoint of the stability of the lipid particles.

The content of the phospholipid relative to 100 parts by mass of the total of the phospholipid and the lysophospholipid in the lipid particles of the present disclosure is preferably 30 parts by mass or more, more preferably 45 parts by mass or more, and still more preferably 50 parts by mass or more, and is preferably 99 parts by mass or less, more preferably 98 parts by mass or less, and still more preferably 97 parts by mass or less from the viewpoint of the stability of the lipid particles.

The content of the lysophospholipid relative to 100 parts by mass of the total of the phospholipid and the lysophospholipid in the lipid particles of the present disclosure is preferably 1 part by mass or more, more preferably 2 parts by mass or more, and still more preferably 3 parts by mass or more, and is preferably 70 parts by mass or less, more preferably 55 parts by mass or less, and still more preferably 40 parts by mass or less from the viewpoint of the stability of the lipid particles.

A lipid particle is, for example, an amorphous solid lipid particle, liposome, bicelle, or micelle. A liposome has a spherical shape having at least one lipid bilayer structure (lipid bilayer), a bicelle has an oval and flat shape having at least one lipid bilayer, and a micelle is formed of a lipid monolayer (lipid monolayer). The lipid particle in the present disclosure is preferably an amorphous solid lipid particle, liposome, or bicelle, and particularly preferably an amorphous solid lipid particle or a liposome.

The lipid particle of the present disclosure may have a multilamellar structure (multilayer membrane structure) or a unilamellar structure (single membrane structure). The lipid particle preferably has a multilamellar structure from the viewpoint of improving persistence of the effect of a medicinal component described later, and preferably has a unilamellar structure from the viewpoint of improving the transparency of the appearance of the cosmetic composition or the like of the present disclosure.

The membrane structure of the lipid particles of the present disclosure is in any of gel, liquid crystal, and amorphous states. A cosmetic composition or the like containing lipid particles alone in any of the above states tends to have an improved storage stability compared with a cosmetic composition or the like containing lipid particles in any of the above states in combination. Particles with a uniform molecular arrangement can be obtained by uniformly mixing lipid molecules at the molecular level, and the progress of crystallization can be suppressed, resulting in lipid particles with excellent long-term stability. The film structure can be measured by the method described in Examples.

The fluorescence anisotropy of the lipid particles of the present disclosure can be measured by blending a fluorescent substance into the lipid particles, and it can be seen that the lower the fluorescence anisotropy value, the higher the membrane fluidity of the lipid particles. The high membrane fluidity of the lipid particles of the present disclosure promotes transdermal permeability, and the function of replenishing defective sites of intercellular lipids tends to improve. Fluorescence anisotropy can be a physical property that allows observation of the distribution behavior of the particles of the present invention within the skin.

The lipid particle can contain a medicinal component and the like. The lipid particle can be loaded with a hydrophilic component or a hydrophobic component. Normally, when the lipid particle contains a hydrophilic component, the hydrophilic component is contained between a core portion of the lipid particle and a hydrophilic moiety of the lipid bilayer membrane, and when the lipid particle contains a hydrophobic component, the hydrophobic component is incorporated into the lipid bilayer and becomes a membrane component. Examples of the medicinal component include allantoin, carnosine, ascorbic acid, magnesium ascorbate phosphate, ascorbyl tetrahexyldecanoate, L-ascorbic acid 2-glycoside, carotene, retinol, retinol acetate, retinol palmitate, tocopherol acetate, tocopherol, glutathione, arbutin, linoleic acid, linolenic acid, oleic acid, palmitic acid, ubiquinone, inulin, glycyrrhetinic acid, potassium glycyrrhizinate, tranexamic acid, carnitine, water-soluble vitamins and water-soluble vitamin derivatives excluding the above components (specifically, ascorbic acid, magnesium ascorbate phosphate, ascorbyl tetrahexyldecanoate, and L-ascorbic acid 2-glycoside), oil-soluble vitamins and oil-soluble vitamin derivatives excluding the above components (specifically, carotene, retinol, retinol acetate, retinol palmitate, tocopherol acetate, and tocopherol), extracts derived from plants and animals, and extracts derived from minerals. When the lipid particle has a multiple layer structure, among the above-mentioned medicinal components, the water-soluble component can be incorporated between a hydrophilic core portion of the lipid particle and a hydrophilic moiety of the lipid bilayer membrane, and the oil-soluble component can be incorporated into a lipid bilayer membrane whose interior is hydrophobic. The medicinal component contained in the lipid particle is retained in the lipid particle, and, for example, when the medicinal component works on the skin, it can be slowly released inside the skin.

The content of the medicinal component in 100 parts by mass of the lipid particles of the present disclosure is preferably 0 parts by mass or more, more preferably 0.01 parts by mass or more, and still more preferably 0.1 parts by mass or more, and is preferably 30 parts by mass or less, more preferably 20 parts by mass or less, and still more preferably 15 parts by mass or less, from the viewpoint of the stability of the cosmetic composition or the like.

The lipid particles of the present disclosure are blended into a cosmetic composition or the like containing a polyhydric alcohol and water, and may be in a dispersed state as a solution (such as a colloidal solution). The lipid particles may be used in the form of powder when not used in a cosmetic composition or the like. When used in the form of powder, a solution containing lipid particles (such as a colloidal solution) obtained by an appropriate method may be dried to obtain powder. The lipid particles of the present disclosure may be used in the form of powder and added directly to a cosmetic or the like. When the cosmetic or the like contains a polyhydric alcohol and water, the cosmetic or the like containing resin particles added in the form of powder corresponds to the cosmetic composition or the like of the present disclosure.

The content of the lipid particles (or the content of the lipid components) relative to 100 parts by mass of the cosmetic composition or the like of the present disclosure is preferably 0.01 parts by mass or more, more preferably 0.05 parts by mass or more, and still more preferably 0.1 parts by mass or more, and is preferably 20 parts by mass or less, more preferably 15 parts by mass or less, still more preferably 10 parts by mass or less, and still further preferably 8 parts by mass or less, and may be 5 parts by mass or less.

The cosmetic composition or the like of the present disclosure may be produced to have a low content of lipid particles (or lipid components), and then the content of the lipid particles (or lipid components) may be adjusted to an appropriate content through a concentration step. The cosmetic composition or the like of the present disclosure may be also produced to have a high content of lipid particles (or lipid components), and then the content of the lipid particles (or lipid components) may be adjusted to an appropriate content through a dilution step. The appropriate content refers to the above-mentioned content range of the lipid particles (or lipid components).

### <Polyhydric Alcohol>

The polyhydric alcohol in the present disclosure is an alcohol having two or more hydroxyl groups in the molecule. Polyhydric alcohols are classified into dihydric alcohols and trihydric or higher alcohols according to the number of hydroxyl groups. The polyhydric alcohol may be a polyhydric alcohol polymer or other compounds having two or more hydroxyl groups in the molecule. The polyhydric alcohol is preferably an alcohol composed only of three elements C, H and O having two or more hydroxyl groups in the molecule, and more preferably an alcohol composed of a hydrocarbon group having two or more hydroxyl groups in the molecule and a hydroxyl group, or an alcohol composed of a hydrocarbon group, a hydroxyl group, and an ether bond. The polyhydric alcohol preferably does not have a ketone group in the molecule.

A dihydric alcohol is an alcohol having two hydroxyl groups in one molecule, and examples thereof include alkanediol having 2 to 20 carbon atoms, preferably 2 to 10 carbon atoms, such as ethylene glycol, diethylene glycol, triethylene glycol, propylene glycol, 1,3-butylene glycol, 1,2-butanediol, 1,4-butylene glycol, isopentyldiol, 1,2-pentanediol, 1,2-hexanediol, 1,2-octanediol, 1,2-decanediol, and dipropylene glycol. From the viewpoint of suppressing aggregation of lipid particles, the dihydric alcohol is preferably 1,3-butylene glycol, 1,2-pentanediol, isopentyldiol, 1,2-hexanediol, 1,2-octanediol, and 1,2-decanediol, and more preferably 1,3-butylene glycol and 1,2-octanediol. The dihydric alcohol may be also selected from the following polyhydric alcohol polymers and other compounds having two or more hydroxyl groups in the molecule.

A trihydric or higher alcohol is an alcohol having three or more hydroxyl groups in one molecule. Examples of trihydric alcohols include alkanetriols having 3 to 20 carbon atoms, preferably 3 to 10 carbon atoms, such as glycerin, trimethylolpropane, and 1,2,6-hexanetriol. Examples of tetrahydric alcohols include alkanetetraols having 4 to 20 carbon atoms such as pentaerythritol. Examples of pentahydric alcohols include alkanepentaols having 5 to 20 carbon atoms such as xylitol. Examples of hexahydric alcohols include alkanehexaols having 6 to 20 carbon atoms such as sorbitol and mannitol. The trihydric or higher alcohol may be selected from the following polyhydric alcohol polymers or other compounds having two or more hydroxyl groups in the molecule.

Examples of the polyhydric alcohol polymers include diethylene glycol, dipropylene glycol, triethylene glycol, polypropylene glycol, tetraethylene glycol, diglycerin, polyethylene glycol, triglycerin, tetraglycerin, and polyglycerin.

As other compounds having two or more hydroxyl groups in the molecule, glycerin monoalkyl ethers can be given. Examples of the glycerin monoalkyl ethers include xyl alcohol, selachyl alcohol, and batyl alcohol. As other compounds having two or more hydroxyl groups in the molecule, sugar alcohols can be also given. Examples of the sugar alcohols include sorbitol, maltitol, maltotriose, mannitol, sucrose, erythritol, glucose, fructose, starch sugar, maltose, xylytose, and starch sugar reduced alcohol.

These polyhydric alcohols can be used singly or in combination of two or more.

The cosmetic composition or the like of the present disclosure preferably contains the polyhydric alcohol in an amount of 1% by mass or more, more preferably 3% by mass or more, and still more preferably 5% by mass or more, relative to 100% by mass of the total of the cosmetic composition or the like. The cosmetic composition or the like of the present disclosure preferably contains the polyhydric alcohol in an amount of 20% by mass or less, more preferably 18% by mass or less, and still more preferably 15% by mass or less, relative to 100% by mass of the total of the cosmetic composition or the like. When the content of the polyhydric alcohol is within the above range, the storage stability of the cosmetic composition or the like tends to be further improved.

In the cosmetic composition or the like of the present disclosure, the content of the trihydric or higher alcohol is 0 to 20% by mass, preferably 0 to 15% by mass, and particularly preferably 0 to 10% by mass, relative to 100% by mass of the polyhydric alcohol. When the content of the trihydric or higher alcohol is within the above range, the cosmetic composition or the like tends to have good storage stability, less stickiness, and an excellent use feeling. It is also a preferred embodiment to blend the trihydric or higher alcohol into the cosmetic composition or the like, and the cosmetic composition or the like may contain the trihydric or higher alcohol in an amount of 1% by mass or more, preferably 5% by mass or more, and more preferably 10% by mass or more, relative to 100% by mass of the polyhydric alcohol. In the present disclosure, the content of the dihydric alcohol is, relative to 100% by mass of the polyhydric alcohol, preferably 80% by mass or more, more preferably 85% by mass or more, still more preferably 90% by mass or more, and particularly preferably 95% by mass or more, and may be 100% by mass or more.

The content of the polyhydric alcohol (total content of dihydric alcohol and trihydric or higher alcohol) contained in 100 parts by mass of the cosmetic composition or the like of the present disclosure is preferably 0.10 parts by mass or more, more preferably 0.15 parts by mass or more, still more preferably 1 part by mass or more, still further preferably 2 parts by mass or more, and particularly preferably 3 parts by mass or more, and is preferably 35 parts by mass or less, more preferably 25 parts by mass or less, and still more preferably 20 parts by mass or less. When the content of the polyhydric alcohol is within the above range, the storage stability of the cosmetic composition or the like of the present disclosure tends to be further improved.

In the cosmetic composition or the like of the present disclosure, the content of the trihydric or higher alcohol in 100 parts by mass of the total content of the dihydric alcohol and the trihydric or higher alcohol is preferably 0 to 50 parts by mass, more preferably 0 to 40 parts by mass, still more preferably 0 to 30 parts by mass, still further preferably 0 to 25 parts by mass, and particularly preferably 0 to 20 parts by mass. When the content of the trihydric or higher alcohol is within the above range, the cosmetic composition or the like tends to have less stickiness and an excellent use feeling. In the present disclosure, the content of the dihydric alcohol in 100 parts by mass of the total content of the dihydric alcohol and the trihydric or higher alcohol is preferably 50 parts by mass or more, more preferably 60 parts by mass or more, still more preferably 70 parts by mass or more, still further preferably 75 parts by mass or more, and particularly preferably 80 parts by mass or more.

### <Water>

In the present disclosure, water includes normal tap water, ion-exchanged water, pure water, soft water, hard water, natural water, deep seawater, alkaline ion water, and other purified water obtained by various methods. In the present disclosure, water also includes water containing salts and metals, aqueous buffer solutions, and the like. Examples of the aqueous buffer solution include a phosphate buffer solution and a Tris buffer solution. The cosmetic composition or the like of the present disclosure preferably contains water in an amount of 50% by mass or more, more preferably 60% by mass or more, and still more preferably 70% by mass or more, relative to 100% by mass of the total of the cosmetic composition or the like. The cosmetic composition or the like of the present disclosure preferably contains water in an amount of 95% by mass or less, more preferably 90% by mass or less, and still more preferably 85% by mass or less, relative to 100% by mass of the total of the cosmetic composition or the like. When the content of water is within the above range, the storage stability of the cosmetic composition or the like of the present disclosure tends to be further improved.

### <Storage Stability Improving Agent>

The cosmetic composition or the like of the present disclosure may contain a storage stability improving agent. The use of the storage stability improving agent further improves the storage stability of the cosmetic composition or the like. Examples of the storage stability improving agent include alkaline earth metal hydroxides such as calcium hydroxide and magnesium hydroxide; carbonates of alkaline earth metal or ammonia such as calcium carbonate and ammonium carbonate, amines (e.g., alkylamines such as dimethylamine, diethylamine, trimethylamine, and triethylamine; cyclic amines such as morpholine; and alcohol amines such as triethanolamine and diethanolamine), and basic amino acids (e.g., hydroxylysine, lysine, arginine, histidine, tryptophan, and ornithine). Among these, alkaline earth metal hydroxides, alcohol amines, basic amino acid, and the like are preferred.

The amount of the storage stability improving agent contained in the cosmetic composition or the like of the present disclosure is preferably 0.1 ppm (based on mass; hereinafter the same) or more, more preferably 1 ppm or more, and still more preferably 10 ppm or more, and is preferably 1% by mass or less, more preferably 3000 ppm or less, and still more preferably 1000 ppm or less.

Presumably, because the storage stability improving agent enters between the lipid particles, and electrostatic repulsion occurs between the negative charge of the storage stability improving agent and the negative charge on the lipid particle surface, the storage stability of the lipid particles is improved.

### <Other Constituents>

The cosmetic composition or the like of the present disclosure may contain substances other than the above-mentioned lipid particles containing at least a phospholipid, polyhydric alcohol, water, and storage stability improving agent. Such substances include a thickener, a powder component, a pH adjuster, a nonionic surfactant, an anionic surfactant, a cationic surfactant, an amphoteric surfactant, an oil agent, a moisturizing agent, a water-soluble polymer, an antioxidant, an ultraviolet absorber, a chelating agent, an antiseptic agent, an antibacterial agent, a colorant, a fragrance, and the like. As long as the substance is of a type that is usually blended in cosmetics, it can be blended as appropriate. The cosmetic composition or the like of the present disclosure may contain a monohydric alcohol, a dihydric alcohol dialkyl ether, a dihydric alcohol ether ester, or the like.

Examples of the monohydric alcohol of the present disclosure include linear alcohols such as ethanol, propanol, lauryl alcohol, myristyl alcohol, cetyl alcohol, stearyl alcohol, oleyl alcohol, behenyl alcohol, and cetostearyl alcohol; branched alcohols such as isopropanol, isobutyl alcohol, t-butyl alcohol, 2-butyl-1-octanol, 2-hexyl-1-decanol, 2-octyl-1-dodecanol, isostearyl alcohol, 2-decyl-1-tetradecanol, and lanolin alcohol; and alcohols with an ether bond such as ethylene glycol monoalkyl ethers, such as ethylene glycol monomethyl ether, ethylene glycol monoethyl ether, ethylene glycol monobutyl ether, ethylene glycol monophenyl ether, ethylene glycol monohexyl ether, ethylene glycol mono-2-methylhexyl ether, ethylene glycol isoamyl ether, ethylene glycol benzyl ether, and ethylene glycol isopropyl ether. The monohydric alcohol may be a lower alcohol having about 1 to 10 carbon atoms, preferably about 2 to 6 carbon atoms, and may be a higher alcohol having about 11 to 30 carbon atoms, preferably about 11 to 20 carbon atoms. A combination of a lower alcohol and a higher alcohol may also be used. From the viewpoint of the solubility of lipid particles, the monohydric alcohol is preferably a lower alcohol such as ethanol, propanol, isopropanol, and t-butyl alcohol, and more preferably ethanol. From the viewpoint of suppressing aggregation of lipid particles, the monohydric alcohol is preferably a monohydric branched alcohol, more preferably 2-octyl-1-dodecanol, isostearyl alcohol, and 2-decyl-1-tetradecanol, and still more preferably 2-octyl-1-dodecanol and 2-decyl-1-tetradecanol.

Examples of the dihydric alcohol dialkyl ether of the present disclosure include C₂₋₆ alkanediol di-C₁₋₁₀ alkyl ethers such as ethylene glycol dimethyl ether, ethylene glycol diethyl ether, and ethylene glycol dibutyl ether. Ethylene glycol di-C₁₋₆ alkyl ethers are preferred.

Examples of the dihydric alcohol ether ester of the present disclosure include ether compounds of C₂₋₆ alkanediol mono-C₂₋₆ carboxylic acid esters and alcohols having 1 to 10 carbon atoms such as ethylene glycol monomethyl ether acetate, ethylene glycol monoethyl ether acetate, ethylene glycol monobutyl ether acetate, ethylene glycol monophenyl ether acetate, ethylene glycol diadipate, ethylene glycol disuccinate, diethylene glycol monoethyl ether acetate, diethylene glycol monobutyl ether acetate, propylene glycol monomethyl ether acetate, propylene glycol monoethyl ether acetate, propylene glycol monopropyl ether acetate, and propylene glycol monophenyl ether acetate.

The content of the monohydric alcohol, the content of the dihydric alcohol dialkyl ether, and the content of the dihydric alcohol ether ester contained in 100 parts by mass of the cosmetic composition or the like of the present disclosure are each preferably 0.01 parts by mass or more, more preferably 0.05 parts by mass or more, and still more preferably 0.1 parts by mass or more, and are each preferably 20 parts by mass or less, more preferably 15 parts by mass or less, and still more preferably 10 parts by mass or less, from the viewpoint of the stability of the lipid particles.

Examples of the thickener include dextrin, sodium pectate, sodium alginate, PVM (methyl vinyl ether), locust bean gum, tamarind gum, dialkyldimethylammonium sulfate cellulose, aluminum magnesium silicate, bentonite, hectorite, AlMg silicate (veegum), laponite, and anhydrous silicic acid.

The content of the thickener contained in 100 parts by mass of the cosmetic composition or the like of the present disclosure is preferably 0.001 parts by mass or more, more preferably 0.005 parts by mass or more, still more preferably 0.05 parts by mass or more, and is preferably 3 parts by mass or less, more preferably 1 part by mass or less, and still more preferably 0.5 parts by mass or less.

Examples of the powder component include inorganic powder (e.g., talc, kaolin, mica, sericite, muscovite, phlogopite, synthetic mica, lepidolite, biotite, vermiculite, magnesium carbonate, calcium carbonate, aluminum silicate, barium silicate, calcium silicate, magnesium silicate, strontium silicate, metal tungstate, magnesium, silica, zeolite, barium sulfate, calcined calcium sulfate (calcined gypsum), calcium phosphate, fluorine apatite, hydroxyapatite, ceramic powder, metal soap (e.g., zinc myristate, calcium palmitate, and aluminum stearate) and boron nitride)); organic powder (e.g., polyamide resin powder (nylon powder), polyethylene powder, polymethylmethacrylate powder, polystyrene powder, copolymer resin powder of styrene and acrylic acid, benzoguanamine resin powder, polytetrafluoroethylene powder, and cellulose powder); inorganic white pigment (e.g., titanium dioxide and zinc oxide); inorganic red pigment (e.g., iron oxide (colcothar) and iron titanate); inorganic brown pigment (e.g., γ-iron oxide); inorganic yellow pigment (e.g., yellow iron oxide and loess); inorganic black pigment (e.g., black iron oxide and lower titanium oxide); inorganic violet pigment (e.g., manganese violet and cobalt violet); inorganic green pigment (e.g., chrome oxide, chrome hydroxide, and cobalt titanate); inorganic blue pigment (e.g., ultramarine and iron blue); pearl pigment (e.g., titanium oxide coated mica, titanium oxide coated bismuth oxychloride, titanium oxide coated talc, colored titanium oxide coated mica, bismuth oxychloride, and argentine); metal powder pigment (e.g., aluminum powder and copper powder); and organic pigment such as zirconium, barium, and aluminum lake (e.g., Red No. 201, Red No. 202, Red No. 204, Red No. 205, Red No. 220, Red No. 226, Red No. 228, Red No. 405, Orange No. 203, Orange No. 204, Yellow No. 205, Yellow No. 401 and Blue No. 404, Red No. 3, Red No. 104, Red No. 106, Red No. 227, Red No. 230, Red No. 401, Red No. 505, Orange No. 205, Yellow No. 4, Yellow No. 5, Yellow No. 202, Yellow No. 203, Green No. 3, and Blue No. 1); and natural pigment (e.g., chlorophyll and 6-carotene).

Examples of the pH adjuster include a mixture of a hydroxycarboxylic acid and its alkali metal salt such as lactic acid-sodium lactate and citric acid-sodium citrate; a mixture of a dicarboxylic acid and its alkali metal salt such as succinic acid-sodium succinate; an alkali metal hydroxide such as sodium hydroxide and potassium hydroxide, ammonia (may be an aqueous ammonia), citric acid, tartaric acid, lactic acid, phosphoric acid, neutral amino acids (e.g., threonine and cysteine), sodium acyl sarcosinate (sodium lauroylsarcosinate), acyl glutamate, sodium acyl-6-alanine, glutathione, and pyrrolidone carboxylate.

The content of the pH adjuster contained in 100 parts by mass of the cosmetic composition or the like of the present disclosure is preferably 0.001 parts by mass or more, more preferably 0.005 parts by mass or more, and still more preferably 0.01 parts by mass or more, and is preferably 3 parts by mass or less, more preferably 1 part by mass or less, and still more preferably 0.5 parts by mass or less.

Examples of the non-ionic surfactant include POE sorbitan fatty acid esters (e.g., POE sorbitan monooleate, POE sorbitan monostearate, POE sorbitan monooleate, and POE sorbitan tetraoleate); POE sorbitol fatty acid esters (e.g., POE sorbitol monolaurate, POE sorbitol monooleate, POE sorbitol pentaoleate, and POE sorbitol monostearate); POE glycerol fatty acid esters (e.g., POE monooleate, such as POE glycerol monostearate, POE glycerol monoisostearate and POE glycerol triisostearate); POE fatty acid esters (e.g., POE distearate, POE monodioleate, POE monostearate (PEG-20 stearate and the like) and ethylene glycol distearate); POE alkylethers (e.g., POE laurylether, POE oleylether, POE stearylether, POE behenylether, POE-2-octyldodecylether, and POE cholestanolether); Pluronic (registered trademark) surfactants; POE/POP alkyl ethers (e.g., POE/POP cetylether, POE/POP-2-decyltetradecylether, POE/POP monobutylether, POE/POP hydrous lanolin, and POE/POP glycerolether); tetraPOE/tetraPOP-ethylenediamine condensates (e.g., Tetronic); POE castor oil, POE hydrogenated castor oil, and their derivatives (e.g., POE castor oil, POE hydrogenated castor oil, POE hydrogenated castor oil monoisostearate, POE hydrogenated castor oil triisostearate, POE hydrogenated castor oil monopyroglutamic acid-monoisostearic acid diester, and POE-hydrogenated castor oil maleic acid); POE bees wax or POE lanolin derivatives (e.g., POE sorbitol bees wax); alkanolamides (e.g., coconut oil fatty acid diethanolamide, lauric monoethanolamide and fatty acid isopropanolamide); POE propylene glycol fatty acid esters; POE alkylamines; POE fatty acid amides; sucrose fatty acid esters; alkylethoxydimethylamine oxides; trioleylphosphoric acid, sorbitan fatty acid esters (e.g., sorbitan monooleate, sorbitan monoisostearate, sorbitan monolaurate, sorbitan monopalmitate, sorbitan monostearate, sorbitan sesquioleate, sorbitan torioleate, diglycerol sorbitan penta-2-ethylhexylate, and diglycerol sorbitan tetra-2-ethylhexylate); glycerol fatty acids (e.g., glycerol mono-cotton seed oil fatty acid, glycerol monoerucate, glycerol sesquioleate, glycerol monostearate, glycerol α,α′-oleate pyrogluatamate, glycerol monostearate malate, polyglyceryl monoisostearate, and polyglyceryl diisostearate); propylene glycol fatty acid esters (e.g., propylene glycol monostearate); hydrogenated castor oil derivatives; and glycerolalkyl ethers. The “POE” represents a polyethylene glycol residue (when two hydroxyl groups are bonded, it becomes a polyoxyethylene unit, and when one hydroxyl group is bonded, it becomes a polyoxyethylene unit having a hydroxy group at the terminal). The “POP” represents a polypropylene glycol residue (when two hydroxyl groups are bonded, it becomes a polyoxypropylene unit, and when one hydroxyl group is bonded, it becomes a polyoxypropylene unit having a hydroxy group at the terminal). The POE/POP means POE or POP.

Examples of the anionic surfactant include fatty acid soap (e.g., sodium laurate and sodium palmitate); higher alkyl sulfate ester salt (e.g., sodium lauryl sulfate and potassium lauryl sulfate); alkyl ether sulfate ester salt (e.g., POE lauryl sulfate triethanolamine and sodium POE lauryl sulfate); N-acyl sarcosinic acid (e.g., sodium lauroyl sarcocinate); higher fatty acid amide sulfonate (e.g., sodium N-myristoyl-N-methyltaurate, sodium methyl cocoyl taurate, and sodium lauryl methyl taurate); phosphate ester salt (e.g., sodium POE oleylether phosphate and POE stearylether phosphate); sulfosuccinate (e.g., sodium di-2-ethylhexyl sulfosuccinate, sodium monolauroyl monoethanolamide polyoxyethylene sulfosuccinate, and sodium lauryl polypropylene glycol sulfosuccinate); alkylbenzene sulfonate (e.g., sodium linear dodecylbenzene sulfonate, triethanolamine linear dodeylbenzene sulfonate, and linear dodecylbenzene sulfonate); higher fatty acid ester sulfate ester salt (e.g., sodium hydrogenated glyceryl cocoate sulfate); N-acyl glutamate (e.g., monosodium N-lauroyl glutamate, disodium N-stearoyl glutamate, and monosodium N-myristoyl-L-glutamate); sulfonated oil (e.g., Turkey red oil); POE alkyl ether carboxylic acid; POE alkyl aryl ether carboxylate; α-olefine sulfonate; higher fatty acid ester sulfonate; secondary alcohol sulfate ester salt; higher fatty acid alkylolamide sulfate ester salt; sodium lauroyl monoethanolamide succinate; N-palmitoyl asparaginate ditriethanolamine; and sodium casein.

Examples of the cationic surfactant include alkyltrimethyl ammonium salt (e.g., stearyltrimethyl ammonium chloride, and lauryltrimethyl ammonium chloride); alkylpyridinium salt (e.g., cetylpyridinium chloride); distearyldimethyl ammonium chloride dialkyldimethyl ammonium salt; poly (N,N′-dimethyl-3,5-methylenepiperidinium) chloride; alkyl quaternary ammonium salt; alkyldimethylbenzyl ammonium salt; alkylisoquinolinium salt; dialkylmorphonium salt; POE alkylamine; alkylamine salt; polyamine fatty acid derivative; amyl alcohol fatty acid derivative; benzalkonium chloride; and benzethonium chloride.

Examples of the amphoteric surfactant include imidazoline-based amphoteric surfactant (e.g., sodium 2-undecyl-N,N,N-(hydroxyethylcarboxymethyl)-2-imidazoline and 2-cocoyl-2-imidazolinium hydroxide-1-carboxyethyloxy disodium salt); and betaine-based surfactant (e.g., 2-heptadecyl-N-carboxymethyl-N-hydroxyethyl imidazolinium betaine, lauryl dimethylaminoacetic acid betaine, alkyl betaine, amidobetaine, and sulfobetaine).

The content of the surfactant contained in 100 parts by mass of the cosmetic composition or the like of the present disclosure is preferably 0.0001 parts by mass or more, more preferably 0.0005 parts by mass or more, and still more preferably 0.001 parts by mass or more, and is preferably 5 parts by mass or less, more preferably 3 parts by mass or less, and still more preferably 1 part by mass or less.

The oil agent is not particularly limited, but examples thereof include fatty acids, fats and oils, ester oils, silicone oils, and hydrocarbon oils. These components may be used singly or in combination of two or more.

Examples of the fatty acids include lauric acid, myristic acid, palmitic acid, stearic acid, oleic acid, behenic acid, undecylenic acid, lanolin acid, and isostearic acid.

Examples of the fats and oils include coconut oil, palm oil, hydrogenated palm oil, avocado oil, sesame oil, olive oil, kukui nut oil, grape particle oil, safflower oil, almond oil, corn oil, cottonseed oil, sunflower seed oil, grape seed oil, hazelnut oil, macadamia nut oil, meadowfoam oil, and rosehip oil.

Examples of the ester oils include ethyl oleate, isopropyl myristate, isopropyl palmitate, myristyl myristate, cetyl palmitate, oleyl oleate, octyldodecyl myristate, octyldodecyl oleate, ethyl isostearate, isopropyl isostearate, cetyl 2-ethylhexanoate, cetostearyl 2-ethylhexanoate, glyceryl tri-2-ethylhexanoate, caprylic/capric triglyceride, glyceryl triisopalmitate, pentaerythritol tetra-2-ethylhexanoate, isocetyl octanoate, isostearyl octanoate, isocetyl isostearate, octyldodecyl isostearate, and octyldodecyl dimethyloctanoate.

Examples of the silicone oils include methylpolysiloxane, highly polymerized methylpolysiloxane, methylphenylpolysiloxane, octamethyltrisiloxane, decamethyltetrasiloxane, octamethylcyclotetrasiloxane, decamethylcyclopentasiloxane, dodecamethylcyclohexasiloxane, methylcyclopolysiloxane, alcohol-modified silicone, alkyl-modified silicone, amino-modified silicone, and epoxy-modified silicone.

Examples of the hydrocarbon oils include liquid paraffin, olive squalane, rice squalane, squalane, pristane, white petrolatum, paraffin wax, ozokerite, ceresine, and microcrystalline wax.

Examples of the moisturizing agent include chondroitin sulfate, hyaluronic acid, mucoitin sulfate, charonic acid, atelocollagen, cholesteryl-12-hydroxy stearate, sodium lactate, bile salt, dl-pyrrolidone carboxylate, short-chain soluble collagen, diglycerin (EO)PO adducts, extracts of Rosa roxburghii, yarrow extracts, and melilot extracts.

Examples of the water-soluble polymer include plant polymers (e.g., gum arabic, gum tragacanth, galactan, guar gum, carob gum, gum karaya, carrageenan, pectin, agar, quince seed (Cydonia oblonga), algae colloid (brown algae extract), starch (rice, corn, potato, wheat), and glycyrrhizic acid); microorganism polymers (e.g., xanthan gum, dextran, succinoglucan, and pullulan); and animal polymers (e.g., collagen, casein, albumin, and gelatin); starch polymers (e.g., carboxymethylstarch and methylhydroxypropylstarch); cellulose polymers (e.g., methylcellulose, ethylcellulose, methylhydroxypropylcellulose, hydroxyethylcellulose, sodium cellulose sulfate, hydroxypropylcellulose, carboxymethylcellulose, sodium calboxymethyl cellulose, crystalline cellulose, and cellulose powder); alginic acid polymers (e.g., sodium alginate and propylene glycol alginate); vinyl polymers (e.g., polyvinyl alcohol, polyvinylmethyl ether, polyvinylpyrrolidone, and carboxyvinyl polymer (carbomer)); polyoxyethylene polymers (e.g., polyethylene glycol 20,000, polyethylene glycol 40,000, and polyethylene glycol 60,000); acrylic polymers (e.g., sodium polyacrylate, polyethyl acrylate, and polyacrylamide); polyethylene-imine; and cationic polymers.

The content of the water-soluble polymer contained in 100 parts by mass of the cosmetic composition or the like of the present disclosure is preferably 0.001 parts by mass or more, more preferably 0.005 parts by mass or more, and still more preferably 0.05 parts by mass or more, and is preferably 3 parts by mass or less, more preferably 1 part by mass or less, and still more preferably 0.5 parts by mass or less.

Examples of the antioxidant include tocopherols, dibutylhydroxytoluene, butylhydroxyanisole, and gallic acid esters.

Examples of the ultraviolet absorber include benzoic acid ultraviolet absorbers (e.g., para-aminobenzoic acid (hereinafter abbreviated as “PABA”), PABA monoglycerol ester, N,N-dipropoxy-PABA ethyl ester, N,N-diethoxy-PABA ethyl ester, N,N-dimethyl-PABA ethyl ester, N,N-dimethyl-PABA butyl ester, and N,N-dimethyl-PABA ethyl ester); anthranilic acid ultraviolet absorbers (e.g., homomenthyl-N-acetyl anthranilate); salicylic acid ultraviolet absorbers (e.g., amyl salicylate, menthyl salicylate, homomenthyl salicylate, octyl salicylate, phenyl salicylate, benzyl salicylate, and p-isopropanolphenyl salicylate); cinnamic acid ultraviolet absorbers (e.g., octyl cinnamate, ethyl-4-isopropyl cinnamate, methyl-2,5-diisopropyl cinnamate, ethyl-2,4-diisopropyl cinnamate, methyl-2,4-diisopropyl cinnamate, propyl-p-methoxy cinnamate, isopropyl-p-methoxy cinnamate, isoamyl-p-methoxy cinnamate, octyl-p-methoxy cinnamate (2-ethylhexyl-p-methoxy cinnamate), 2-ethoxyethyl-p-methoxy cinnamate, cyclohexyl-p-methoxy cinnamate, ethyl-α-cyano-β-phenyl cinnamate, 2-ethylhexyl-α-cyano-β-phenyl cinnamate, and glycerol mono-2-ethylhexanoyl-diparamethoxy cinnamate); benzophenone ultraviolet absorbers (e.g., 2,4-dihydroxybenzophenone, 2,2′-dihydroxy-4-methoxybenzophenone, 2,2′-dihydroxy-4,4′-dimethoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl-4′-phenylbenzophenone-2-carboxylate, 2-hydroxy-4-n-octoxybenzophenone, and 4-hydroxy-3-carboxybenzophenone); 3-(4′-methylbenzylidene)-d,L-camphor, 3-benzylidene-d,l-camphor; 2-phenyl-5-methylbenzoxazole; 2,2′-hydroxy-5-methylphenylbenzotriazole; 2-(2′-hydroxy-5′-t-octylphenyl)benzotriazole; 2-(2′-hydroxy-5′-methylphenyl)benzotriazole; dibenzalazine; dianisoylmethane; 4-methoxy-4′-t-butyldibenzoylmethane, and 5-(3,3-dimethyl-2-norbornylidene)-3-pentan-2-one.

Examples of the chelating agent include 1-hydroxyethane-1,1-diphosphonic acid, tetrasodium 1-hydroxyethane-1,1-diphosphonate, disodium edetate, trisodium edetate, tetrasodium edetate, sodium citrate, sodium polyphosphate, sodium metaphosphate, gluconic acid, phosphoric acid, citric acid, ascorbic acid, succinic acid, edetic acid, and trisodium hydroxyethyl ethylenediamine triacetate.

Examples of the antiseptic agent and the antibacterial agent include parabens such as ethylparaben, isopropylparaben, butylparaben, benzylparaben, and sodium salts thereof, benzoic acid, benzoic acid salts, alkyldiaminoethylglycine hydrochloride, photosensitizer, chlorocresol, chlorobutanol, salicylic acid, salicylic acid salts, sorbic acid and salts thereof, dehydroacetic acid and salts thereof, trichloro hydroxydiphenyl ether (also known as triclosan), phenoxyethanol, phenol, sodium lauryldiaminoethylglycine, resorcin, zinc-ammonia-silver composite substituted zeolite, pantothenyl ethyl ether benzoate, isopropyl methylphenol, cetylpyridinium chloride, benzalkonium chloride, benzethonium chloride, chlorhexidine hydrochloride, orthophenyl phenol, sodium orthophenyl phenol, silver-copper zeolite, chlorhexidine gluconate, cresol, chloramine T, chloroxylenol, chlorphenesin, chlorhexidine, 1,3-dimethylol-5,5-dimethylhydantoin, alkylisoquinolinium bromide, thianthol, and thymol.

The content of the antiseptic agent and/or the antibacterial agent contained in 100 parts by mass of the cosmetic composition or the like of the present disclosure is preferably 0.001 parts by mass or more, more preferably 0.01 parts by mass or more, and still more preferably 0.05 parts by mass or more, and is preferably 5 parts by mass or less, more preferably 3 parts by mass or less, and still more preferably 1 part by mass or less.

Examples of the fragrance include terpenes and terpenoids such as citral, menthol, camphor, salvinorin A, cannabinoid, hinokitiol, limonene, farnesol, and vitamin A; aromatic alcohols such as phenoxyethanol; phenols such as eugenol and shogaol; esters such as butyric acid esters and propionic acid esters; lactones such as γ-nonalactone and γ-undecalactone; and aldehydes each having 6 to 20 carbon atoms. Components that can be classified as both polyhydric alcohols and fragrances are classified as fragrances in the present invention.

The content of the other constituents relative to 100% by mass of the cosmetic composition or the like of the present disclosure is preferably 0% by mass or more and 20% by mass or less, and more preferably 0% by mass or more and 10% by mass or less. The other constituents can be used singly or in combination of two or more. Among the other constituents, for those whose specific amounts are specified above, it is preferable to use the amounts specified above.

<Physical Properties of the Cosmetic Composition or the Like of the Present disclosure>

The lipid particles of the present disclosure preferably have an average particle size of 10 nm or more, more preferably 30 nm or more, and further preferably 50 nm or more, 80 nm or more, 100 nm or more, and 120 nm or more in this order. The upper limit of the average particle size is preferably 600 nm or less, and more preferably 500 nm or less, 400 nm or less, 300 nm or less, 200 nm or less, and 180 nm or less in this order. When the average particle size is within the above range, the cosmetic composition or the like of the present disclosure tends to have further improved storage stability, further improved transdermal permeability, and improved skin barrier properties.

The lipid particles of the present disclosure preferably have an average particle size of 150 nm or more and 600 nm or less, more preferably 150 nm or more and 500 nm or less, and still more preferably 160 nm or more and 400 nm or less. When the average particle size is within the above range, the cosmetic composition or the like of the present disclosure tends to have further improved storage stability.

The lipid particles of the present disclosure preferably have a polydispersity index of 0.5 or less, more preferably 0.4 or less, still more preferably 0.3 or less, and particularly preferably 0.25 or less. When the polydispersity index is within the above range, the cosmetic composition or the like of the present disclosure tends to have further improved storage stability. In addition, the transdermal permeability tends to be further improved, and the skin barrier properties tend to be improved.

The average particle size of the present disclosure refers to an average particle size obtained by using the Einstein-Stokes relation by the dynamic light scattering method. The polydispersity index of the present disclosure can be measured based on the cumulant method.

In the cosmetic composition or the like of the present disclosure, the rate of change in the average particle size after storage at 25° C. for 1 month ((average particle size after storage/average particle size before storage−1)×100(%)) is preferably ±30% or less. In the cosmetic composition or the like of the present disclosure, the rate of change in the average particle size after storage at 25° C. for 1 month is more preferably ±20% or less, still more preferably ±18% or less, still further preferably ±15% or less, and particularly preferably ±10% or less. When the rate of change in the average particle size after storage at 25° C. for 1 month is within the above range, the cosmetic composition or the like of the present disclosure has excellent long-term storage stability and has commercial utility.

The average particle size and the polydispersity index of the lipid particles can be measured by the method described in Examples.

In the cosmetic composition or the like of the present disclosure, the polydispersity index of the lipid particles after long-term storage (for example, storage at 4° C., 25° C., 40° C. or 50° C. for 1 month, 3 months or 6 months) is preferably 0.5 or less, more preferably 0.4 or less, still more preferably 0.3 or less, and particularly preferably 0.25 or less. When the polydispersity index of the lipid particles after long-term storage is within the above range, the cosmetic composition or the like of the present disclosure has excellent long-term storage stability and has commercial utility. In particular, the rate of change in polydispersity index before and after long-term storage ((polydispersity index after storage/polydispersity index before storage−1)×100 (%)) is preferably ±25% or less, more preferably ±20% or less, and still more preferably ±15% or less.

As the particle size distribution of the lipid particles of the present disclosure, from the viewpoint of suppressing aggregation, the abundance ratio of lipid particles having a diameter of 80 nm or more and 400 nm or less is preferably 70 area % or more and 100 area % or less, more preferably 75 area % or more and 99 area % or less, and still more preferably 80 area % or more and 98 area % or less. From the viewpoint of the feel of the cosmetic composition or the like, the abundance ratio of lipid particles having a diameter of 1000 nm or more (coarse particles) is preferably 30 area % or less, more preferably 20 area % or less, and still more preferably 15 area % or less. The abundance ratio in the particle size distribution of the lipid particles of the present disclosure can be calculated from a scattering intensity distribution obtained through analysis by the cumulant method using, for example, FPAR-1000 (manufactured by Otsuka Electronics Co., Ltd.).

In the cosmetic composition or the like of the present disclosure, the abundance ratio of lipid particles having a diameter more than 1000 nm after long-term storage (for example, storage at 4° C., 25° C., 40° C. or 50° C. for 1 month, 3 months or 6 months) is preferably 30 area % or less, more preferably 20 area % or less, and still more preferably 15 area % or less. When the abundance ratio of lipid particles having a diameter more than 1000 nm after long-term storage is within the above range, the cosmetic composition or the like of the present disclosure has excellent long-term storage stability and has commercial utility.

The cosmetic composition or the like of the present disclosure preferably has a pH of, for example, 3.0 to 10.0, more preferably 3.5 to 9.5.

The change value of pH (pH before storage−pH after storage) of the cosmetic composition or the like of the present disclosure after long-term storage (for example, storage at 4° C., 25° C., 40° C. or 50° C. for 1 month, 3 months or 6 months) is preferably ±1.2 or less, more preferably ±1.0 or less, and still more preferably ±0.8 or less. When the change value of pH is within the above range, the cosmetic composition or the like of the present disclosure has excellent long-term storage stability and has commercial utility.

The appearance of the cosmetic composition or the like of the present disclosure is preferably in a uniform state without precipitation, deposition, or layer separation, and is preferably in a uniform state without precipitation, deposition, or layer separation even after long-term storage (for example, storage at 4° C., 25° C., 40° C. or 50° C. for 1 month, 3 months or 6 months), as before the storage. The cosmetic composition or the like of the present disclosure has the above appearance properties, and thus is excellent in long-term storage stability and has commercial utility.

The cosmetic composition or the like of the present disclosure has a rich texture, that is a unique feeling derived from the lipid particles. When the cosmetic composition or the like contains liposomes as the lipid particles, it further has a rich texture, that is a unique feeling derived from the liposomes. In addition, the cosmetic composition or the like of the present disclosure has no stickiness feeling or is less sticky.

Furthermore, the cosmetic composition or the like of the present disclosure has excellent permeability into epidermis and dermis.

Transdermal permeability, such as permeability to epidermis and dermis, can be evaluated in such a manner that a cosmetic composition or the like including lipid particles containing a label compound such as a water-soluble fluorescent dye such as carboxyfluorescein and carboxyfluorescein derivatives, an oil-soluble fluorescent dye such as DiI (1,1′-dioctadecyl-3,3,3′,3′-tetramethylindocarbocyanine perchlorate) and DiO (3,3′-dioctadecyloxacarbocyanine perchlorate), a medicinal component, a specific lipid, and a fluorescent labeling substance is applied to a three-dimensional skin model, human skin, or the like to perform a skin permeability test. The permeability of the label compound can be evaluated by analyzing a solution remaining in the upper part of the skin or skin model after the permeability test, a solution permeated the lower part of the skin or skin model after the permeability test, and the skin after the permeability test. The label compound in the solution remaining in the upper part and the solution permeated the lower part of the skin or skin model after the permeability test can quantified by various methods. The skin after the permeability test can be evaluated by a method of performing image analysis of a section of the skin to evaluate the degree of permeation of the label compound, a method of extracting the label compound from the collected skin and quantifying them by various analytical techniques, or a method of collecting each horny layer from the collected skin by tape stripping to perform image analysis or to extract the label compound and quantify them by various analytical techniques. For the image analysis of a section of the skin, a confocal laser scanning microscope, a fluorescence microscope, an optical microscope, and a small-angle and wide-angle x-ray scattering can be used. Quantitation method of the label compound includes a method using a combination of a detection method such as ultraviolet-visible spectrophotometer, fluorescence spectrophotometer, differential refractive index, mass spectrometer, electrical conductivity, evaporative light scattering, or corona charged aerosol, and an instrument such as liquid chromatography or gas chromatography, depending on the type of the label compound; and a method using a label compound quantitation kit.

The zeta potential of the particle surface of the lipid particles of the present disclosure is preferably −30 mV or less, more preferably −40 mV or less, and still more preferably −50 mV or less. When the zeta potential is within the above range, the storage stability of the cosmetic composition or the like of the present disclosure tends to be further improved. The zeta potential can be measured by the method described in Examples.

The particle surface of the lipid particles of the present disclosure exhibits a positive or negative zeta potential, and the absolute value of the zeta potential is preferably 5 mV or more, more preferably 10 mV or more, still more preferably 20 mV or more, and particularly preferably 30 mV or more. When the zeta potential of the particle surface is a positive value and is within the above range, the cosmetic composition or the like of the present disclosure tends to have further improved transdermal permeability. When the zeta potential of the particle surface is a negative value and is within the above range, the cosmetic composition or the like of the present disclosure tends to have further improved storage stability. The zeta potential can be measured by the method described in Examples.

The solid content concentration of lipid particles in the cosmetic composition or the like used for measurement of a zeta potential is preferably less than 0.5% by mass.

Another aspect of the present invention includes a cosmetic composition for storage or cosmetic quasi-drug for storage including lipid particles containing at least a phospholipid, a polyhydric alcohol, and water, wherein the content of a trihydric or higher alcohol in the polyhydric alcohol is 0 to 20% by mass relative to 100% by mass of the polyhydric alcohol.

Another aspect of the present invention includes a method for storing a cosmetic composition including lipid particles containing at least a phospholipid, a polyhydric alcohol, and water at 0 to 60° C. (preferably 4 to 50° C.) for 1 to 36 months (preferably 1 to 6 months) to use it, wherein the content of a trihydric or higher alcohol in the polyhydric alcohol is 0 to 20% by mass relative to 100% by mass of the polyhydric alcohol.

[Production Method of the Cosmetic Composition or the Like of the Present disclosure]

A production method of the cosmetic composition or the like of the present disclosure is not particularly limited. The production method preferably has a step of mixing a phospholipid, a polyhydric alcohol, water, and optionally a medicinal component to produce a composition containing those (hereinafter also referred to as a “mixing step”).

Another aspect of the present invention includes a production method of a cosmetic composition or the like having a mixing step of mixing a solution containing a phospholipid and a polyhydric alcohol and a solution containing water, wherein, in the mixing step, the content of a trihydric or higher alcohol in the polyhydric alcohol is 0 to 20% by mass relative to 100% by mass of the polyhydric alcohol.

The production method of the cosmetic composition or cosmetic quasi-drug of the present disclosure preferably has a step of mixing a lipid solution containing a phospholipid and a polyhydric alcohol (solution 1) and a solution containing water (solution 2) using a device having a flow channel structure such as a microfluidic device.

In the device having a flow channel structure of the present disclosure, the mixing ratio (mass of solution 1/mass of solution 2) of the lipid solution containing a phospholipid and a polyhydric alcohol (solution 1) and the solution containing water (solution 2) is preferably 99/1 to 1/99, more preferably 50/1 to 1/50, and still more preferably 25/1 to 1/25.

The lipid solution (solution 1) of the present disclosure preferably contains a phospholipid and a polyhydric alcohol, and more preferably contains, in addition to them, a sterol and a lysophospholipid. The lipid solution (solution 1) may further contain a ceramide, and may further contain a monohydric alcohol. For the phospholipid, polyhydric alcohol, sterol, lysophospholipid, ceramide, and monohydric alcohol, reference is made to the above description.

The lipid solution (solution 1) of the present disclosure may contain a medicinal component and may contain a component exhibiting oil solubility (oil-soluble component). The medicinal component is as described above, and the medicinal component to be used may be varied depending on the composition of the lipid solution.

The solution containing water (solution 2) of the present disclosure may contain a water-soluble component in addition to water. Examples of the water soluble component include a water-soluble polymer, a monohydric alcohol, a dihydric alcohol, a trihydric or higher alcohol, a storage stability improving agent, and a pH adjuster. For the water-soluble polymer, monohydric alcohol, dihydric alcohol, trihydric or higher alcohol, storage stability improving agent, and pH adjuster, reference is made to the above description.

The solution containing water (solution 2) of the present disclosure may contain a medicinal component and may contain a component exhibiting water solubility (water-soluble component). The medicinal component is as described above, and the medicinal component to be used may be varied depending on the composition of the solution containing water.

The content of the phospholipid in 100 parts by mass of the lipid solution (solution 1) of the present disclosure is preferably 0.1 parts by mass or more, more preferably 0.5 parts by mass or more, and still more preferably 1 part by mass or more, and is preferably 95 parts by mass or less, more preferably 90 parts by mass or less, and still more preferably 80 parts by mass or less.

The content of the polyhydric alcohol in 100 parts by mass of the lipid solution (solution 1) of the present disclosure is preferably 5 parts by mass or more, more preferably 10 parts by mass or more, and still more preferably 15 parts by mass or more, and is preferably 99.9 parts by mass or less, more preferably 99.5 parts by mass or less, and still more preferably 99 parts by mass or less.

The content of the sterol in 100 parts by mass of the lipid solution (solution 1) of the present disclosure is preferably 0.01 parts by mass or more, more preferably 0.05 parts by mass or more, and still more preferably 0.1 parts by mass or more, and is preferably 10 parts by mass or less, more preferably 9.5 parts by mass or less, and still more preferably 9 parts by mass or less.

The content of the lysophospholipid in 100 parts by mass of the lipid solution (solution 1) of the present disclosure is preferably 0.01 parts by mass or more, more preferably 0.02 parts by mass or more, and still more preferably 0.03 parts by mass or more, and is preferably 20 parts by mass or less, more preferably 15 parts by mass or less, and still more preferably 10 parts by mass or less.

The content of the monohydric alcohol in 100 parts by mass of the lipid solution (solution 1) of the present disclosure is preferably 0 parts by mass or more, more preferably 0.1 parts by mass or more, and still more preferably 1 part by mass or more, and is preferably 99 parts by mass or less, more preferably 90 parts by mass or less, and still more preferably 80 parts by mass or less.

When the lipid solution (solution 1) of the present disclosure contains a medicinal component, the content of the medicinal component in 100 parts by mass of the lipid solution (solution 1) is preferably 0 parts by mass or more, more preferably 0.01 parts by mass or more, and still more preferably 0.1 parts by mass or more, and is preferably 50 parts by mass or less, more preferably 40 parts by mass or less, and still more preferably 30 parts by mass or less.

The content of water in 100 parts by mass of the solution containing water (solution 2) of the present disclosure is preferably 50 parts by mass or more, more preferably 75 parts by mass or more, and still more preferably 90 parts by mass or more, and may be 100 parts by mass.

When the solution containing water (solution 2) of the present disclosure contains a water-soluble component, the content of the water-soluble component in 100 parts by mass of the solution containing water (solution 2) is preferably 50 parts by mass or less, more preferably 25 parts by mass or less, and still more preferably 10 parts by mass or less, and may be 0 parts by mass.

The content of the water-soluble polymer in 100 parts by mass of the solution containing water (solution 2) of the present disclosure may be 0 parts by mass or more, 0.01 parts by mass or more, or 0.02 parts by mass or more, and may be 20 parts by mass or less, 15 parts by mass or less, or 10 parts by mass or less.

The content of the monohydric alcohol in 100 parts by mass of the solution containing water (solution 2) of the present disclosure may be 0 parts by mass or more, 0.01 parts by mass or more, or 0.1 parts by mass or more, and may be 50 parts by mass or less, 25 parts by mass or less, or 10 parts by mass or less.

The content of the dihydric alcohol in 100 parts by mass of the solution containing water (solution 2) of the present disclosure may be 0 parts by mass or more, 0.01 parts by mass or more, or 0.1 parts by mass or more, and may be 50 parts by mass or less, 25 parts by mass or less, or 10 parts by mass or less.

The content of the trihydric (or higher) alcohol in 100 parts by mass of the solution containing water (solution 2) of the present disclosure may be 0 parts by mass or more, 0.01 parts by mass or more, or 0.1 parts by mass or more, and may be 50 parts by mass or less, 25 parts by mass or less, or 10 parts by mass or less.

When the solution containing water (solution 2) of the present disclosure contains a medicinal component, the content of the medicinal component in 100 parts by mass of the solution containing water (solution 2) is preferably 0 parts by mass or more, more preferably 0.01 parts by mass or more, and still more preferably 0.1 parts by mass or more, and is preferably 50 parts by mass or less, more preferably 40 parts by mass or less, and still more preferably 30 parts by mass or less.

The mixing step may be performed using a disperser having a mechanical stirring structure, such as a homogenizer, and preferably performed using a device having a flow channel structure, such as a microfluidic device. When the cosmetic composition or the like of the present disclosure is produced using a microfluidic device, the storage stability of the cosmetic composition or the like tends to further improve. Although the microfluidic device is not particularly limited, the device described in WO2018/190423 can be given as an example thereof. The microfluidic device is preferably a micromixer for mixing two or more liquids, and the micromixer is preferably a baffle mixer. The micromixer has a structure for mixing liquids passing through microfluidic channels. The baffle mixer has a structure in which a baffle is arranged in a micro-sized flow channel. A phospholipid is usually self-assembled to form lipid particles in the mixing step, but a formation method of lipid particles is not particularly limited. The mixing step may be performed at room temperature, and may also be performed under a heating condition or cooling condition. The mixing step may be performed under normal pressure, increased pressure, or reduced pressure.

The production method of the present disclosure may include other steps such as a purification step, a concentration step, and a dilution step, in addition to the mixing step.

When the lipid particles of the present disclosure are used as a powder, the cosmetic composition or the like produced may be dried to obtain a powder. Examples of the drying method of the present disclosure include freeze drying, vacuum drying, air drying, spray drying, and flash distillation. Among these, freeze drying is preferred. Drying may be performed in combination with a general concentration method. As the general concentration method, a distillation method such as atmospheric distillation, reduced-pressure distillation, molecular distillation, and flash distillation can also be used. A membrane separation method such as ultrafiltration and centrifugation can also be used.

### [Use of the Cosmetic Composition or the Like of the Present Disclosure]

The cosmetic composition or the like of the present disclosure is less sticky, has an excellent use feeling, and has excellent long-term storage stability, and thus can be suitably applied to skin cosmetics, skin external preparations, and the like.

The term “cosmetic” in the present disclosure means using as cosmetics, quasi-drugs, and pharmaceutical drugs, which are directly applied to the skin of the human body, and the cosmetics include skin cosmetics, skin external preparations, hair cosmetics, hair external preparations, and the like. The cosmetic composition or the like in the present disclosure is used for the above-mentioned applications. The cosmetic composition may be used as a cosmetic as it is, or may be used as an additive for a cosmetic (raw material of a cosmetic).

Another aspect of the present invention includes cosmetics including a composition including lipid particles containing at least a phospholipid, a polyhydric alcohol, and water, wherein the content of a trihydric or higher alcohol in the polyhydric alcohol is 0 to 20% by mass relative to 100% by mass of the polyhydric alcohol.

Another aspect of the present invention includes a production method of cosmetics made by blending or using a composition including lipid particles containing at least a phospholipid, a polyhydric alcohol, and water, wherein the content of a trihydric or higher alcohol in the polyhydric alcohol is 0 to 20% by mass relative to 100% by mass of the polyhydric alcohol.

Furthermore, another aspect of the present invention includes use, as cosmetics, of a composition including lipid particles containing at least a phospholipid, a polyhydric alcohol, and water, wherein the content of a trihydric or higher alcohol in the polyhydric alcohol is 0 to 20% by mass relative to 100% by mass of the polyhydric alcohol.

### [Production Method of Lipid Particles of the Present Disclosure]

A production method of the lipid particles of the present disclosure is not particularly limited, and the lipid particles of the present disclosure can be produced, for example, by the method described in the above production method of the cosmetic composition or the like.

Another aspect of the present invention includes a production method of lipid particles having a mixing step of mixing a solution containing a phospholipid and a polyhydric alcohol and a solution containing water, wherein, in the mixing step, the content of a trihydric or higher alcohol in the polyhydric alcohol is 0 to 20% by mass relative to 100% by mass of the polyhydric alcohol.

The production method of the lipid particles of the present disclosure preferably has a step of mixing a lipid solution containing a phospholipid and a polyhydric alcohol (solution 1) and a solution containing water (solution 2) using a device having a flow channel structure such as a microfluidic device.

In the device having a flow channel structure of the present disclosure, the mixing ratio (mass of solution 1/mass of solution 2) of the lipid solution containing a phospholipid and a polyhydric alcohol (solution 1) and the solution containing water (solution 2) is preferably 99/1 to 1/99, more preferably 50/1 to 1/50, and still more preferably 25/1 to 1/25.

The lipid solution (solution 1) of the present disclosure preferably contains a phospholipid and a polyhydric alcohol, and more preferably contains, in addition to them, a sterol and a lysophospholipid. The lipid solution (solution 1) may further contain a ceramide, and may further contain a monohydric alcohol. For the phospholipid, polyhydric alcohol, sterol, lysophospholipid, ceramide, and monohydric alcohol, reference is made to the above description.

The lipid solution (solution 1) of the present disclosure may contain a medicinal component and may contain a component exhibiting oil solubility (oil-soluble component). The medicinal component is as described above, and the medicinal component to be used may be varied depending on the composition of the lipid solution.

The solution containing water (solution 2) of the present disclosure may contain a water-soluble component in addition to water. Examples of the water-soluble component include a water-soluble polymer, a monohydric alcohol, a dihydric alcohol, a trihydric or higher alcohol, a storage stability improving agent, and a pH adjuster. For the water-soluble polymer, monohydric alcohol, dihydric alcohol, trihydric or higher alcohol, storage stability improving agent, and pH adjuster, reference is made to the above description.

The solution containing water (solution 2) of the present disclosure may contain a medicinal component and may contain a component exhibiting water solubility (water-soluble component). The medicinal component is as described above, and the medicinal component to be used may be varied depending on the composition of the solution containing water.

The content of the phospholipid in 100 parts by mass of the lipid solution (solution 1) of the present disclosure is preferably 0.1 parts by mass or more, more preferably 0.5 parts by mass or more, and still more preferably 1 part by mass or more, and is preferably 95 parts by mass or less, more preferably 90 parts by mass or less, and still more preferably 80 parts by mass or less.

The content of the polyhydric alcohol in 100 parts by mass of the lipid solution (solution 1) of the present disclosure is preferably 5 parts by mass or more, more preferably 10 parts by mass or more, and still more preferably 15 parts by mass or more, and is preferably 99.9 parts by mass or less, more preferably 99.5 parts by mass or less, and still more preferably 99 parts by mass or less.

The content of the sterol in 100 parts by mass of the lipid solution (solution 1) of the present disclosure is preferably 0.01 parts by mass or more, more preferably 0.05 parts by mass or more, and still more preferably 0.1 parts by mass or more, and is preferably 10 parts by mass or less, more preferably 9.5 parts by mass or less, and still more preferably 9 parts by mass or less.

The content of the lysophospholipid in 100 parts by mass of the lipid solution (solution 1) of the present disclosure is preferably 0.01 parts by mass or more, more preferably 0.02 parts by mass or more, and still more preferably 0.03 parts by mass or more, and is preferably 20 parts by mass or less, more preferably 15 parts by mass or less, and still more preferably 10 parts by mass or less.

The content of the monohydric alcohol in 100 parts by mass of the lipid solution (solution 1) of the present disclosure is preferably 0 parts by mass or more, more preferably 0.1 parts by mass or more, and still more preferably 1 part by mass or more, and is preferably 99 parts by mass or less, more preferably 90 parts by mass or less, and still more preferably 80 parts by mass or less.

When the lipid solution (solution 1) of the present disclosure contains a medicinal component, the content of the medicinal component in 100 parts by mass of the lipid solution (solution 1) is preferably 0 parts by mass or more, more preferably 0.01 parts by mass or more, and still more preferably 0.1 parts by mass or more, and is preferably 50 parts by mass or less, more preferably 40 parts by mass or less, and still more preferably 30 parts by mass or less.

The content of water in 100 parts by mass of the solution containing water (solution 2) of the present disclosure is preferably 50 parts by mass or more, more preferably 75 parts by mass or more, and still more preferably 90 parts by mass or more, and may be 100 parts by mass.

When the solution containing water (solution 2) of the present disclosure contains a water-soluble component, the content of the water-soluble component in 100 parts by mass of the solution containing water (solution 2) is preferably 50 parts by mass or less, more preferably 25 parts by mass or less, and still more preferably 10 parts by mass or less.

The content of the water-soluble polymer in 100 parts by mass of the solution containing water (solution 2) of the present disclosure may be 0 parts by mass or more, 0.01 parts by mass or more, or 0.02 parts by mass or more, and may be 20 parts by mass or less, 15 parts by mass or less, or 10 parts by mass or less.

The content of the monohydric alcohol in 100 parts by mass of the solution containing water (solution 2) of the present disclosure may be 0 parts by mass or more, 0.01 parts by mass or more, or 0.1 parts by mass or more, and may be 50 parts by mass or less, 25 parts by mass or less, or 10 parts by mass or less.

The content of the dihydric alcohol in 100 parts by mass of the solution containing water (solution 2) of the present disclosure may be 0 parts by mass or more, 0.01 parts by mass or more, or 0.1 parts by mass or more, and may be 50 parts by mass or less, 25 parts by mass or less, or 10 parts by mass or less.

The content of the trihydric (or higher) alcohol in 100 parts by mass of the solution containing water (solution 2) of the present disclosure may be 0 parts by mass or more, 0.01 parts by mass or more, or 0.1 parts by mass or more, and may be 50 parts by mass or less, 25 parts by mass or less, or 10 parts by mass or less.

When the solution containing water (solution 2) of the present disclosure contains a medicinal component, the content of the medicinal component in 100 parts by mass of the solution containing water (solution 2) is preferably 0 parts by mass or more, more preferably 0.01 parts by mass or more, and still more preferably 0.1 parts by mass or more, and is preferably 50 parts by mass or less, more preferably 40 parts by mass or less, and still more preferably 30 parts by mass or less.

The present application claims priority based on Japanese Patent Application No. 2020-189158 filed on Nov. 13, 2020. All the contents described in Japanese Patent Application No. 2020-189158 filed on Nov. 13, 2020 are incorporated herein by reference.

### EXAMPLES

Hereinafter, the invention will be described in detail with reference to examples. However, the invention should not be limited to these examples. Unless otherwise mentioned, the term “part(s)” means “part(s) by mass” and “%” means “% by mass”.

Measurements in Examples (Production Examples) were each performed as follows.

### <pH Measurement>

A lipid particle solution described in each Examples was measured with pH Electrode 9615S (manufactured by Horiba, Ltd.).

### <Viscosity Measurement>

A lipid particle solution described in each Examples was measured with Viscometer Model BM II (manufactured by Toki Sangyo Co., Ltd.) using Rotor No. 3 under the conditions of a rotation speed of 6 rpm and 25° C.

### <Phase Transition Temperature Measurement>

A few mg of a lipid particle solution described in each Examples was weighed in an aluminum pan and measured with DSC3500 (manufactured by Netsch). The temperature is raised from −10° C. to 90° C. at a rate of 2° C./min, and the temperature at which an endothermic peak is confirmed is defined as a phase transition temperature of a lipid particle. If the phase transition temperature cannot be confirmed under the above measurement conditions, the temperature is lowered from 25° C. to −10° C. at a rate of 10° C./min, and the temperature at which an exothermic peak is confirmed is defined as a phase transition temperature of the lipid particle.

### <Measurements of Average Particle Size, Polydispersity Index, and Content of Coarse Particles>

A cosmetic composition (lipid particle solution) described in each Example was diluted 100-fold with pure water, and a blended composition (skin lotion, formulated product) prepared in each Example was diluted 100-fold with pure water. These diluted compositions were kept warm for 30 minutes in a constant temperature bath at 25° C., and then measured at 25° C. using FPAR-1000 (manufactured by Otsuka Electronics Co., Ltd.) by the dynamic light scattering method. The average particle size of the present disclosure refers to an average particle size obtained by using the Einstein-Stokes relation by the dynamic light scattering method. The polydispersity index of the present disclosure can be obtained through analysis by the cumulant method.

### <Zeta Potential Measurement>

A lipid particle solution described in each Example was diluted 100-fold with pure water, and the diluted solution was kept warm for 30 minutes in a constant temperature bath at 25° C. and then measured at 25° C. using Zetasizer Nano ZSP (manufactured by Malvern).

### <Stability Test>

A cosmetic composition (lipid particle solution) and a blended composition (skin lotion, formulated product) described in each Example were each charged into a 30 mL glass container and stored at 40° C., 25° C., 40° C., or 50° C. for 0.25 months, 1 month, 3 months, or 6 months. Appearance, pH, average particle size and polydispersity index of each composition after storage were measured.

### <Electron Microscope Observation>

A transmission electron microscope H-7600 (manufactured by Hitachi, Ltd.) and a scanning electron microscope S-4800 (manufactured by Hitachi, Ltd.) were used. As pretreatment for electron microscope observation, an ice embedding method, a negative staining method, a freeze fracture method, or the like can be used. An observation kit such as K-kit manufactured by Hitachi High-Tech Corporation can also be used. In Examples, samples were pretreated by an ice embedding method using VITROBOT (manufactured by Thermo Fisher Scientific K.K.) or a negative staining method.

### <Evaluation of Use Feeling>

A use feeling test was conducted with eight professional evaluation panelists. An appropriate amount (about 20 μl) of a lipid particle solution or a blended composition was applied to the back of the hand, and one or more items of smoothness, skin penetration, rich texture, skin freshness sensation, and stickiness during application, and moisture retention and persistence of skin resilience after application were judged. The evaluation and judgment criteria for each item in the use feeling evaluation are as described below.

### [Evaluation]

5: Very good

### 4: Good

### 3: Normal

2: Not very good
1: Not good

### [Judgment]

∘: Average score 4 or more
Δ: Average score 3.0 or more and less than 4.0 [symbol]
: Average score less than 3.0

Each of the judgment results showed the characteristic feeling as follows.

### <<Smoothness>>

In the case of ∘, during spreading the applied solution, the solution spread smoothly and evenly, and spread easily without sticking. In the case of [symbol] , sticking and stiffness were felt.

### <<Skin Penetration>>

In the case of ∘, there was a feeling such that the applied solution was tight-fitting to the skin. In the case of [symbol] , there was no feeling such that the applied solution was tight-fitting to the skin.

### <<Rich Texture>>

In the case of ∘, there was a rich texture peculiar to lipid particles. In the case of [symbol] , there was no rich texture peculiar to lipid particles.

### <<Stickiness>>

In the case of ∘, no stickiness was felt when the solution was applied. In the case of [symbol] , stickiness was felt.

### <<Moisture Retention>>

In the case of ∘, the skin after the elapse of 2 hours after application had a moisturizing feeling. In the case of [symbol] , no moisturizing feeling remained.

### <<Persistence of Skin Resilience>>

In the case of ∘, the skin after the elapse of 2 hours from application felt such flexible elasticity that the skin was pushed back from the inside thereof. In the case of [symbol] , no flexible elasticity was felt.

The judgment criteria for skin freshness sensation are as described below.

### <<Skin Freshness Sensation>>

A case of having a skin freshness sensation upon application was judged as “yes”, and a case of having no skin freshness sensation was judged as “no”.

### <Fluorescence Intensity Measurement>

Using a multi-grading microplate reader SH-9000Lab (manufactured by Corona Electric Co., Ltd.), an appropriate amount of a sample was measured at 30° C. on a 96-well FluoroNunc plate with excitation light of 490 nm and fluorescence of 530 nm. When calculating the amount of fluorescent dye contained in a fluorescent dye-containing lipid particle solution as in Production Example 9 described later, the solution was mixed with polyoxyethylene(10) octylphenyl ether (manufactured by Fujifilm Wako Pure Chemical Corporation, hereinafter abbreviated as Triton X-100), and the fluorescence intensity of the eluted fluorescent dye was measured.

### <Quantification of Caffeine>

Analysis was performed using a liquid chromatography (manufactured by Shimadzu Corporation) and a column (CAPCELL PAK C18 SG120, 4.6 mm I.D.×250 mm, manufactured by Osaka Soda Co., Ltd.). A caffeine solution of already known concentrations was prepared using a developing solution to draw a calibration curve in advance.

UV: 254 nm

Developing solution: water:methanol:acetic acid=39:60:1 (vol/vol)

Column temperature: 45° C.

Injected amount: 5 μL

Flow rate: 0.8 ml/min

### <Quantitative Measurement 1 of Components Contained in Cosmetic Composition (Lipid Particle Solution)>

Analysis was performed using a liquid chromatography (manufactured by Shimadzu Corporation) and a column (CAPCELL PAK C18 SG120, 4.6 mm I.D.×250 mm, manufactured by Osaka Soda Co., Ltd.). A chloroform solution containing components contained in a cosmetic composition at already known concentrations was used to draw a calibration curve in advance.

ELSD: 350 to 360 kPa, 40° C., GAIN 6

UV: 254 nm

Developing solution: methanol 100 vol %

Column temperature: 45° C.

Injected amount: 5 μL

Flow rate: 0.75 ml/min

A cosmetic composition of unknown concentrations was diluted 40-fold with methanol and heated at 50° C. for 5 minutes, filtration was then performed using a membrane filter, and the filtrate solution was measured. As to ELSD retention times of typical components, the retention time of phospholipid is 12.5 to 13.5 minutes and 15.5 to 17.0 minutes, the retention time of lysophospholipid is 4.0 to 6.5 minutes, the retention time of cholesterol is 11.5 to 12.5 minutes, and the retention time of ceramide NG is 12.5 to 13.5 minutes.

### <Quantitative Measurement 2 of Components Contained in Cosmetic Composition (Lipid Particle Solution)>

Analysis was performed using a gas chromatography (GC-2010 PLUS manufactured by Shimadzu Corporation) and a column (DB-WAX manufactured by Agilent J&W, inner diameter 0.25 mm, length 30 m, film thickness 0.25 μm). A 1-pentanol solution containing components contained in the cosmetic composition at already known concentrations was prepared to draw a calibration curve in advance. The internal standard substance is dimethyl sulfoxide at 1000 ppm.

Injection volume: 1.0 μL

Vaporization chamber temperature: 250° C.

Injection Mode: split

Total flow rate: 38.9 mL/min

Column flow rate: 1.16 mL/min

Purge flow rate: 3.0 mL/min

Split ratio: 30.0

Carrier gas: helium

Column temperature: holding at 80° C. for 10 minutes, raising a temperature to 240° C. at a rate of 10° C./min, and holding for 5 minutes

Detector: FID

Detector temperature: 250° C.

Inlet: split

Inlet temperature: 250° C.

A cosmetic composition of unknown concentrations was diluted 80-fold with methanol or 1-pentanol and heated at 50° C. for 5 minutes, filtration was then performed using a membrane filter, and the filtrate solution was measured. As to FID retention times of typical components, the retention time of ethanol is 1.5 to 2.5 minutes, the retention time of 1,3-butanediol is 18.0 to 19.0 minutes, and the retention time of dimethyl sulfoxide, which is the internal standard substance, is 15.5 to 16.5 minutes.

### <Quantitative Measurement of Water Contained in Cosmetic Composition (Lipid Particle Solution)>

Measurement was performed by coulometric titration using a Karl Fischer moisture meter (CA-310 manufactured by Nittoseiko Analytech Co., Ltd.). The amount of water contained in super-dehydrated methanol (manufactured by Fujifilm Wako Pure Chemical Corporation) was measured in advance.

Anolyte: Aquamicron AX (manufactured by Mitsubishi Chemical Corporation)

Catholyte: Aquamicron CXU (manufactured by Mitsubishi Chemical Corporation)

A cosmetic composition of unknown concentrations was diluted 300-fold with super-dehydrated methanol and heated at 50° C. for 5 minutes, and the resulting solution was measured. The amount of water contained in the super-dehydrated methanol measured in advance was subtracted from the measurement results as a background to calculate the amount of water contained in the cosmetic composition of unknown concentrations.

### Production Example 1

A lipid solution (solution 1) obtained by dissolving 6 parts of Phospholipon 90H (manufactured by Lipoid) and 4 parts of SLP-PC70HS (manufactured by Tsuji Oil Mill Co., Ltd.) in 7 parts of 1,3-butanediol (manufactured by Daicel Corporation) and 83 parts of water (solution 2) were mixed in a flow channel structure having two flow channels as described in Example 1 of WO2018/190423 to prepare a lipid particle solution (cosmetic composition 1 of the present disclosure).

### Production Examples 2 to 6

Lipid particle solutions (cosmetic compositions 2 to 6 of the present disclosure) were prepared in the same manner as in Production Example 1, except that the formulation was changed to the formulations (unit: part) as shown in Table 1.

Phospholipon 80H (manufactured by Lipoid) and SLP-PC92H (manufactured by Tsuji Oil Mill Co., Ltd.) were used.

### Production Example 7

A lipid solution (solution 1) obtained by dissolving 6 parts of Phospholipon 90H (manufactured by Lipoid) and 4 parts of SLP-PC70HS (manufactured by Tsuji Oil Mill Co., Ltd.) in 30 parts of glycerin (Sakamoto Yakuhin Kogyo Co., Ltd.) and 60 parts of water (solution 2) were mixed in a flow channel structure having two flow channels as described in Example 1 of WO2018/190423 to prepare a lipid particle solution (comparative cosmetic composition 7).

### Production Example 8

A lipid solution (solution 1) obtained by dissolving 4.8 parts of Phospholipon 90H (manufactured by Lipoid) and 3.2 parts of SLP-PC70HS (manufactured by Tsuji Oil Mill Co., Ltd.) in 10.7 parts of 1,3-butanediol (manufactured by Daicel Corporation) and 21.3 parts of glycerin (Sakamoto Yakuhin Kogyo Co., Ltd.) and 60 parts of water (solution 2) were mixed in a flow channel structure having two flow channels as described in Example 1 of WO20181190423 to prepare a lipid particle solution (comparative cosmetic composition 8).

Table 1 shows the formulations (unit: part) of the lipid particle solutions prepared in Production Examples 1 to 8. Table 2 shows the quantitative measurement results (unit: %) of various components and water contained in the lipid particle solutions prepared in Production Examples 1 to 8.

TABLE 1

| | Production | Production | Production | Production | Production | Production | Production | Production |
|---|---|---|---|---|---|---|---|---|
| Component | Example 1 | Example 2 | Example 3 | Example 4 | Example 5 | Example 6 | Example 7 | Example 8 |
| Phospholipon90H | 6 | 4.8 | 0 | 0 | 0 | 5 | 6 | 4.8 |
| SLP-PC70HS | 4 | 3.2 | 0 | 0 | 0 | 0 | 4 | 3.2 |
| Phospholipon80H | 0 | 0 | 10 | 8 | 7 | 0 | 0 | 0 |
| SLP-PC92H | 0 | 0 | 0 | 0 | 0 | 5 | 0 | 0 |
| 1,3-butanediol | 7 | 9 | 7 | 9 | 10 | 7 | 0 | 10.7 |
| Ethanol | 0 | 0 | 0 | 0 | 0 | 0 | 0 | 0 |
| Glycerin | 0 | 0 | 0 | 0 | 0 | 0 | 30 | 21.3 |
| Water | 83 | 83 | 83 | 83 | 83 | 83 | 60 | 60 |

TABLE 2

| Quantitative | | | | | | | | |
|---|---|---|---|---|---|---|---|---|
| measurement of | | | | | | | | |
| various components | | | | | | | | |
| contained in the lipid | Production | Production | Production | Production | Production | Production | Production | Production |
| particle solutions | Example 1 | Example 2 | Example 3 | Example 4 | Example 5 | Example 6 | Example 7 | Example 8 |
| Phospholipid | 10.1 | 8.0 | 9.9 | 8.1 | 7.1 | 10.2 | 10.0 | 8.1 |
| 1,3-butanediol | 7.1 | 9.0 | 6.9 | 9.0 | 10.0 | 7.2 | — | 10.6 |
| Water | 84.0 | 83.7 | 83.1 | 82.8 | 84.0 | 83.7 | 58.9 | 60.5 |

Table 3 shows the physical properties, appearances, and stabilities of the lipid particle solutions prepared in Production Examples 1 to 8.

TABLE 3

| Storage | Physical | Production | Production | Production | Production | Production | Production | Production | Production |
|---|---|---|---|---|---|---|---|---|---|
| conditions | properties | Example 1 | Example 2 | Example 3 | Example 4 | Example 5 | Example 6 | Example 7 | Example 8 |
| Immediately | pH | 4.6 | 4.7 | 5.1 | 5.1 | 5.1 | 5.1 | 4.6 | 4.5 |
| after | Phase transition | 52.6 | 52.1 | 52.4 | 52.6 | 52.8 | 51.9 | 52.6 | 55.0 |
| preparation | temperature (° C.) | | | | | | | | |
| | Average particle | 358 | 250 | 375 | 283 | 255 | 255 | 658 | 660 |
| | size (nm) | | | | | | | | |
| | Polydispersity | 0.25 | 0.23 | 0.23 | 0.21 | 0.25 | 0.22 | 0.57 | 0.51 |
| | index | | | | | | | | |
| | Zeta potential | −56.6 | −53.0 | −65.2 | −63.8 | −62.1 | −57.4 | — | −0.2 |
| | (mV) | | | | | | | | |
| | Appearance | White | White | Pale yellow | Pale yellow | Pale yellow | White | White | White |
| | | Turbid | Translucent | Turbid | Translucent | Transparent | Translucent | Turbid | Turbid |
| Stored at 4° C. | pH | 4.5 | 4.7 | 5.1 | 5.0 | 4.9 | 5.1 | 3.1 | 2.9 |
| for 1 month | Average particle | 378 | 255 | 384 | 284 | 255 | 257 | 2115 | 1015 |
| | size (nm) | | | | | | | | |
| | Polydispersity | 0.19 | 0.23 | 0.21 | 0.20 | 0.25 | 0.22 | 0.61 | 0.52 |
| | index | | | | | | | | |
| | Appearance | No change | No change | No change | No change | No change | No change | Layer | Layer |
| | | from the | from the | from the | from the | from the | from the | separation | separation |
| | | state im- | state im- | state im- | state im- | state im- | state im- | | |
| | | mediately | mediately | mediately | mediately | mediately | mediately | | |
| | | after | after | after | after | after | after | | |
| | | prepara- | prepara- | prepara- | prepara- | prepara- | prepara- | | |
| | | tion | tion | tion | tion | tion | tion | | |
| Stored at 25° C. | pH | 4.2 | 4.4 | 4.9 | 4.7 | 4.9 | 5.1 | 2.8 | 2.5 |
| for 1 month | Average particle | 398 | 266 | 370 | 293 | 264 | 249 | 3467 | 1548 |
| | size (nm) | | | | | | | | |
| | Polydispersity | 0.19 | 0.23 | 0.22 | 0.21 | 0.23 | 0.23 | 0.65 | 0.61 |
| | index | | | | | | | | |
| | Appearance | No change | No change | No change | No change | No change | No change | Layer | Layer |
| | | from the | from the | from the | from the | from the | from the | separation | separation |
| | | state im- | state im- | state im- | state im- | state im- | state im- | | |
| | | mediately | mediately | mediately | mediately | mediately | mediately | | |
| | | after | after | after | after | after | after | | |
| | | prepara- | prepara- | prepara- | prepara- | prepara- | prepara- | | |
| | | tion | tion | tion | tion | tion | tion | | |
| Stored at 4° C. | pH | 4.5 | 4.5 | 4.8 | 4.9 | 4.9 | 4.9 | — | — |
| for 6 months | Average particle | 380 | 263 | 366 | 273 | 255 | 250 | — | — |
| | size (nm) | | | | | | | | |
| | Polydispersity | 0.21 | 0.22 | 0.19 | 0.24 | 0.25 | 0.23 | — | — |
| | index | | | | | | | | |
| | Appearance | No change | No change | No change | No change | No change | No change | — | — |
| | | from the | from the | from the | from the | from the | from the | | |
| | | state im- | state im- | state im- | state im- | state im- | state im- | | |
| | | mediately | mediately | mediately | mediately | mediately | mediately | | |
| | | after | after | after | after | after | after | | |
| | | prepara- | prepara- | prepara- | prepara- | prepara- | prepara- | | |
| | | tion | tion | tion | tion | tion | tion | | |

From the results in Table 3, it was found that the lipid particle solutions of the present invention have excellent long-term stability.

In the use feeling evaluation of the lipid particle solutions of Production Examples 1 to 6, the judgments on stickiness were ∘, and it was confirmed that no stickiness was felt.

In the use feeling evaluation of Production Examples 7 and 8, the judgments on stickiness were [symbol] , the judgments on rich texture were A, and only a slight rich texture peculiar to the lipid particles was felt.

### <Blended Compositions 1 to 4>

The lipid particle solutions obtained in Production Examples 1 to 4 were blended (unit: part) according to the formulations shown in Table 4, to prepare skin lotions (blended compositions 1 to 4 of the present disclosure).

TABLE 4

| | Blended | Blended | Blended | Blended |
|---|---|---|---|---|
| Component (ingredient) | composition 1 | composition 2 | composition 3 | composition 4 |
| Lipid particle solution of | 1 | 0 | 0 | 0 |
| Production Example 1 | | | | |
| Lipid particle solution of | 0 | 1 | 0 | 0 |
| Production Example 2 | | | | |
| Lipid particle solution of | 0 | 0 | 1 | 0 |
| Production Example 3 | | | | |
| Lipid particle solution of | 0 | 0 | 0 | 1 |
| Production Example 4 | | | | |
| Ultrapure water | 88.513 | 88.513 | 88.513 | 88.513 |
| (Purified water (obtained by | | | | |
| “Milli-Q (registered trademark)” | | | | |
| manufactured by Merck) | | | | |
| 1,3-butanediol | 8 | 8 | 8 | 8 |
| (manufactured by | | | | |
| KH Neochem Co., Ltd.) | | | | |
| Glycerin | 2 | 2 | 2 | 2 |
| (manufactured by | | | | |
| Sakamoto Yakuhin Kogyo Co., Ltd.) | | | | |
| Xanthan gum | 0.05 | 0.05 | 0.05 | 0.05 |
| (manufactured by Sansho Co., Ltd.) | | | | |
| Carbomer | 0.1 | 0.1 | 0.1 | 0.1 |
| (manufactured by Lubrizol) | | | | |
| Sodium hydroxide | 0.037 | 0.037 | 0.037 | 0.037 |
| (manufactured by Fujifilm Wako Pure | | | | |
| Chemical Corporation) | | | | |
| Phenoxyethanol | 0.3 | 0.3 | 0.3 | 0.3 |
| (manufactured by Yokkaichi Chemical | | | | |
| Company Limited) | | | | |

All of the obtained blended compositions immediately after the preparation were homogeneous and had no precipitation observed.

Table 5 shows the physical properties and storage stabilities of the obtained blended compositions.

TABLE 5

| Storage | Physical | Blended | Blended | Blended | Blended |
|---|---|---|---|---|---|
| conditions | properties | composition 1 | composition 2 | composition 3 | composition 4 |
| Immediately | pH | 6.67 | 6.65 | 6.67 | 6.67 |
| after | Average particle | 372 | 309 | 351 | 343 |
| preparation | size (nm) | | | | |
| | Polydispersity | 0.17 | 0.24 | 0.18 | 0.16 |
| | index | | | | |
| | Appearance | Slightly | Slightly | Slightly white | Slightly white |
| | | yellowish white | yellowish white | | |
| Stored at 4° C. | pH | 6.69 | 6.67 | 6.70 | 6.69 |
| for 1 month | Average particle | 453 | 316 | 417 | 319 |
| | size (nm) | | | | |
| | Polydispersity | 0.20 | 0.23 | 0.19 | 0.18 |
| | index | | | | |
| Stored at 25° C. | pH | 6.69 | 6.69 | 6.69 | 6.68 |
| for 1 month | Average particle | 473 | 331 | 404 | 357 |
| | size (nm) | | | | |
| | Polydispersity | 0.23 | 0.15 | 0.19 | 0.17 |
| | index | | | | |

From the results in Table 5, it was found that the skin lotions containing the lipid particle solutions of the present invention have excellent long-term stability.

### <Electron Microscope Observation>

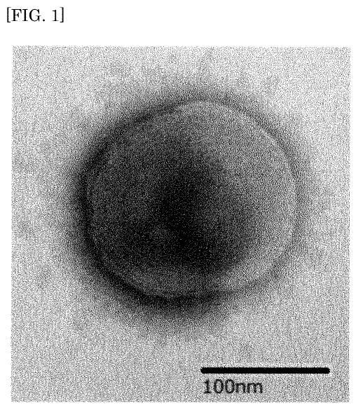

After pretreatment by a negative staining method, a lipid particle in the lipid particle solution of Production Example 2 was observed with a transmission electron microscope. The obtained image is shown in FIG. 1.

From the results of FIG. 1, it was found that the lipid particle contained in the lipid particle solution of Production Example 2 is spherical. It was also found that the lipid particle has a multi-layered structure.

### Production Example 9: Preparation of Fluorescent Dye-Containing Lipid Particle Solution for Skin Permeability Test

A lipid solution (solution 1) obtained by dissolving 6 parts of Phospholipon 90H (manufactured by Lipoid) and 4 parts of SLP-PC70HS (manufactured by Tsuji Oil Mill Co., Ltd.) in 7 parts of 1,3-butanediol (manufactured by Daicel Corporation) and 83 parts of an aqueous solution of 5(6)-carboxyfluorescein (manufactured by Sigma-Aldrich, hereinafter abbreviated as fluorescent dye) dissolved so as to be 0.1 M (solution 2) were mixed in a flow channel structure having two flow channels as described in Example 1 of WO2018/190423 to prepare a fluorescent dye-containing lipid particle solution (cosmetic composition 9 of the present disclosure). The obtained fluorescent dye-containing lipid particle solution was subjected to gel filtration to remove fluorescent dyes in the outer water layer, which were not contained in the interiors of the lipid particles. Sephadex G-50 (manufactured by Cytiva) was used as a filter medium gel, and a pH 7.5 solution obtained by dissolving 0.1 M sodium chloride in a 20 mM tris(hydroxymethyl)aminomethane hydrochloride aqueous solution (hereinafter referred to as Tris buffer solution) was used as an eluent. The resulting fluorescent dye-containing lipid particle solution had an average particle size of 324 nm and a polydispersity index of 0.243. The fluorescent dye concentration was 7.1×10⁻⁴ M.

### <Skin Permeability Test Using Three-Dimensional Skin Model>

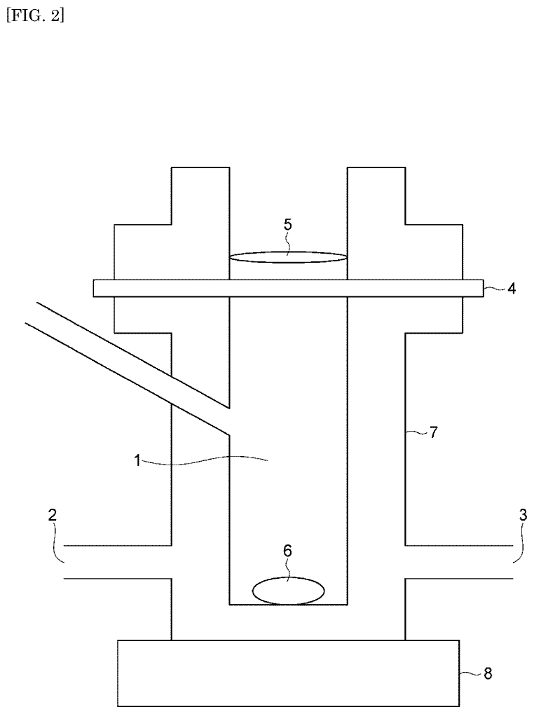

A skin permeability test was performed using the fluorescent dye-containing lipid particle solution prepared in Preparation Example 9 and the Tris buffer solution (pH 7.5) as sample solutions. LSE-high (human skin full-thickness model) commercialized by Roman Industries Co. Ltd. was used as a three-dimensional skin model. The three-dimensional skin model was cut out from the bottom of a transwell of LSE-high. As shown in FIG. 2, a three-dimensional skin model 4 was attached to a vertical Franz diffusion cell (manufactured by Roman Industries Co. Ltd.) (indicated by 7 in FIG. 2) (effective diffusion area: 1.13 cm²) with the dermis side of the three-dimensional skin model 4 facing a receiver solution 1 side, and a sample liquid 5 was applied to the epidermal layer side of the three-dimensional skin model 4. In FIG. 2, a constant temperature circulating water inlet 2 and a constant temperature circulating water outlet 3 were connected to a constant temperature bath, and constant temperature water of 37° C. was flowed from the constant temperature circulating water inlet 2 toward the constant temperature circulating water outlet 3 and circulated around the outer periphery of the vertical Franz diffusion cell 7. The application volume of each sample solution was 1 mL per 1 cm² of effective diffusion area. 3.7 mL of Tris acid buffer (pH 7.5) was used as the receiver solution. During the test, the receiver solution 1 was stirred by rotating a stirrer 6 placed in the receiver solution 1 with a magnetic stirrer 8. The skin permeability test was performed in a laboratory at room temperature of 25° C. and humidity of 60%. The receiver solution 1 was sampled at 0.25 mL each 30 minutes, 120 minutes, 240 minutes, and 360 minutes after application of each sample solution. After completion of each sampling, 0.25 mL of a fresh Tris buffer solution (pH 7.5) was added to each receiver phase. After 120 minutes, 240 minutes, and 360 minutes of the application of the sample solution, some of the three-dimensional skin models 4 were removed from the vertical Franz diffusion cell 7, and frozen blocks were prepared according to the method described in “Master of Cell/Tissue Staining (Yodosha) 2018”. From the prepared frozen blocks, cryosections of 7 sm in thickness were produced at a sample stage temperature of −80° C. and a knife temperature of −21° C. using Cryostar NX70 (manufactured by Thermo Scientific). Fluorescence observation of each cryosection was performed using a fluorescence microscope (ECLIPSE Ti2; manufactured by Nikon Solutions Co., Ltd.).

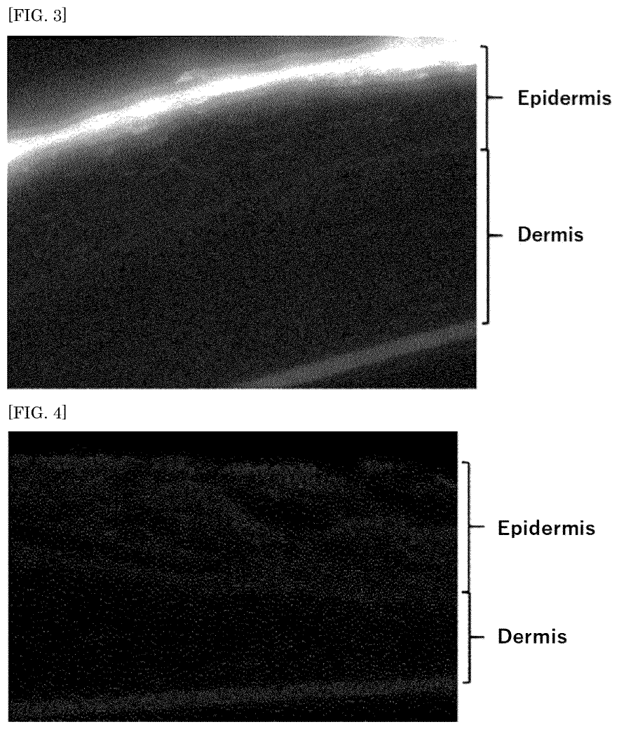

As shown in FIG. 3, in the skin permeability test using the fluorescent dye-containing lipid particle solution as a sample solution, fluorescence was observed in the epidermis and dermis. On the other hand, as shown in FIG. 4, in the test using the Tris buffer solution as a sample solution, fluorescence was not observed in both the epidermis and dermis.

The results of FIGS. 3 and 4 suggest that, in the test group of the fluorescent dye-containing lipid particle solution, the lipid particle solution containing the fluorescent dye permeated the epidermis and dermis of the three-dimensional skin model.

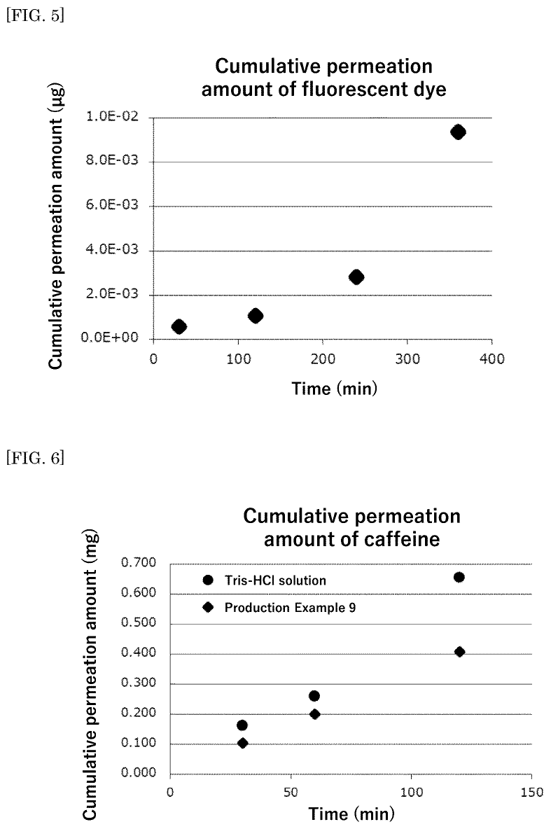

In the skin permeability test using the fluorescent dye-containing lipid particle solution as a sample liquid, the receiver solution 1 obtained at each sampling time was treated with Triton X-100, and fluorescence intensity was measured. The results are shown in FIG. 5.

As shown in FIG. 5, in the test group of the fluorescent dye-containing lipid particle solution, since fluorescence was observed in the receiver solution, it was found that the fluorescent dye contained in lipid particles permeated the epidermis and dermis of the three-dimensional skin model and permeated to the receiver solution under the dermis.

### <Skin Barrier Properties Evaluation Test Using Three-Dimensional Skin Model>

A skin barrier properties evaluation test was performed using the fluorescent dye-containing lipid particle solution prepared in Preparation Example 9 and the Tris buffer solution (pH 7.5) as sample solutions. LSE-high (human skin full-thickness model) commercialized by Roman Industries Co. Ltd. was used as a three-dimensional skin model. The three-dimensional skin model was cut out from the bottom of a transwell of LSE-high. As shown in FIG. 2, a three-dimensional skin model 4 was attached to a vertical Franz diffusion cell (manufactured by Roman Industries Co. Ltd.) (indicated by 7 in FIG. 2) (effective diffusion area: 1.13 cm²) with the dermis side of the three-dimensional skin model 4 facing a receiver solution 1 side, and a sample liquid 5 was applied to the epidermal layer side of the three-dimensional skin model 4. In FIG. 2, a constant temperature circulating water inlet 2 and a constant temperature circulating water outlet 3 were connected to a constant temperature bath, and constant temperature water of 37° C. was flowed from the constant temperature circulating water inlet 2 toward the constant temperature circulating water outlet 3 and circulated around the outer periphery of the vertical Franz diffusion cell 7. The application volume of each sample solution was 1 mL per 1 cm² of effective diffusion area. 3.7 mL of Tris acid buffer (pH 7.5) was used as the receiver solution. During the test, the receiver solution 1 was stirred by rotating a stirrer 6 placed in the receiver solution 1 with a magnetic stirrer 8. The skin barrier properties evaluation test was performed in a laboratory at room temperature of 25° C. and humidity of 60%. After 120 minutes of the application of each sample solution, the applied sample solution was removed, and a Tris buffer solution (pH 7.5) containing 10 mg/ml of caffeine (manufactured by Fujifilm Wako Pure Chemical Corporation) was applied. The receiver solution 1 was sampled at 0.25 mL each 30 minutes, 60 minutes, and 120 minutes after application of the caffeine solution. After completion of each sampling, 0.25 mL of a fresh Tris buffer solution (pH 7.5) was added to each receiver phase.

The receiver solution 1 obtained at each sampling time was diluted 3 to 10-fold with a Tris-HCl solution, and the amount of caffeine in the solution was quantified by liquid chromatography. The results are shown in FIG. 6. It was found that the cumulative permeation amount of caffeine in the receiver solution 1 of the test group of the fluorescent dye-containing lipid particle solution was smaller than the cumulative permeation amount of caffeine in the receiver solution 1 of the test group of the Tris buffer solution. Probably, advance application of the lipid particle solution improved the barrier properties of the three-dimensional skin model and reduced the amount of permeation of caffeine that had been applied later.

### Production Example 10

A lipid solution (solution 1) obtained by dissolving 7.2 parts of Phospholipon 90H (manufactured by Lipoid), 1.5 parts of Cholesterol NF-PW-(JP) (manufactured by Croda Japan K.K.), 1.2 parts of SLP-PC70HS (manufactured by Tsuji Oil Mill Co., Ltd.), and 3.0 parts of Ceramide TIC-001 (manufactured by Takasago International Corporation) in 43.2 parts of 1,3-butanediol (manufactured by Daicel Corporation) and 43.2 parts of ethanol (manufactured by Japan Alcohol Corporation), and 496.5 parts of water (solution 2) were mixed in a baffle mixer type flow channel structure, which is a micromixer, to prepare a lipid particle solution (cosmetic composition 10 of the present disclosure).

### Production Examples 11 to 26

Using ingredients listed in Table 6, lipid particle solutions (cosmetic compositions 11 to 18 of the present disclosure and comparative cosmetic compositions 19 to 26) were prepared in the same manner as in Production Example 10, except that the formulation was changed to the formulations (unit: part) as shown in Tables 7 and 8. Tables 9 and 10 show the quantitative measurement results (unit: %) of various components and water contained in the lipid particle solutions prepared in Production Examples 10 to 26.

TABLE 6

| Component | Product name | Manufacturer |
|---|---|---|
| Hydrogenated lecithin (hydrogenated | Phospholipon90H | Lipoid GmbH |
| soybean phosphatidylcholine) | | |
| Cholesterol | Cholesterol NF-PW-(JP) | Croda Japan K.K. |
| Hydrogenated lysolecithin | SLP-LPC70H | Tsuji Oil Mill Co., Ltd. |
| (hydrogenated soybean | | |
| lysophospholipid) | | |
| N-stearoyl dihydrosphingosine | Ceramide TIC-001 | Takasago International Corporation |
| (ceramide NG) | | |
| Decyltetradecanol | Risonol 24SP | Kokyu Alcohol Kogyo. Co., Ltd. |
| Octanediol | 1,2-octanediol | Tokyo Chemical Industry Co., Ltd. |
| PEG-20 stearate | EMALEX 820 | Nihon Emulsion Co., Ltd. |
| Polyoxyethylene hydrogenated | EMALEX HC-20 | Nihon Emulsion Co., Ltd. |
| castor oil | | |
| Oleyl alcohol | Oleyl alcohol VP | Kokyu Alcohol Kogyo. Co., Ltd. |
| 1,3-butanediol | 1,3-butanediol | Daicel Corporation |
| Ethanol | Ethanol | Japan Alcohol Corporation |
| Glycerin | Concentrated glycerin | Sakamoto Yakuhin Kogyo Co., Ltd. |
| | for cosmetics | |
| Xanthan gum | KELTROL CG-BT | Sansho Co., Ltd. |
| Carbomer | Carbopol 980 | Lubrizol |
| Sodium hydroxide | Sodium Hydroxide | Fujifilm Wako Pure Chemical |
| | | Corporation |
| Phenoxyethanol | Phenoxyethanol-S | Yokkaichi Chemical Company Limited |

TABLE 7

| | | Production | Production | Production | Production | Production | Production | Production | Production | Production |
|---|---|---|---|---|---|---|---|---|---|---|
| | | Example | Example | Example | Example | Example | Example | Example | Example | Example |
| | Component | 10 | 11 | 12 | 13 | 14 | 15 | 16 | 17 | 18 |
| Solution 1 | Hydrogenated | 7.2 | 7.2 | 7.2 | 7.2 | 7.2 | 7.2 | 7.2 | 7.2 | 7.2 |
| | lecithin | | | | | | | | | |
| | Cholesterol | 1.5 | 1.5 | 1.5 | 1.5 | 1.5 | 1.5 | 1.5 | 1.5 | 1.5 |
| | Hydrogenated | 1.2 | 1.2 | 0.3 | 3.0 | 0.6 | 0.6 | 1.2 | 1.2 | 1.2 |
| | lysolecithin | | | | | | | | | |
| | N-stearoyl | 3.0 | 3.0 | 3.0 | 3.0 | 3.0 | 3.0 | 3.0 | 3.0 | 3.0 |
| | dihydrosphingosine | | | | | | | | | |
| | Decyltetradecanol | — | — | — | — | 0.5 | 0.5 | 0.5 | — | — |
| | Octanediol | — | — | — | — | — | — | — | 1.0 | 2.0 |
| | PEG-20 stearate | — | — | — | — | — | — | — | — | — |
| | Polyoxyethylene | — | — | — | — | — | — | — | — | — |
| | hydrogenated | | | | | | | | | |
| | castor oil | | | | | | | | | |
| | Oleyl alcohol | — | — | — | — | — | — | — | — | — |
| | 1,3-butanediol | 43 | 52 | 44 | 43 | 43 | 52 | 43 | 0 | 0 |
| | Ethanol | 43 | 35 | 44 | 43 | 43 | 35 | 43 | 86 | 85 |
| Solution 2 | Water | 497 | 497 | 499 | 499 | 496 | 496 | 499 | 499 | 499 |
| | Total | 596 | 596 | 598 | 598 | 595 | 595 | 599 | 598 | 598 |

TABLE 8

| | | Production | Production | Production | Production | Production | Production | Production | Production |
|---|---|---|---|---|---|---|---|---|---|
| | | Example | Example | Example | Example | Example | Example | Example | Example |
| | Component | 19 | 20 | 21 | 22 | 23 | 24 | 25 | 26 |
| Solution 1 | Hydrogenated | 7.2 | 7.2 | 7.2 | 7.2 | 7.2 | 7.2 | 7.2 | 7.2 |
| | lecithin | | | | | | | | |
| | Cholesterol | 1.5 | 1.5 | 1.5 | 1.5 | 1.5 | 1.5 | 1.5 | 1.5 |
| | Hydrogenated | — | — | — | — | — | — | — | — |
| | lysolecithin | | | | | | | | |
| | N-stearoyl | 3.0 | 3.0 | 3.0 | 3.0 | 3.0 | 3.0 | 3.0 | 3.0 |
| | dihydrosphingosine | | | | | | | | |
| | Decyltetradecanol | — | — | — | — | — | — | — | — |
| | Octanediol | — | — | — | — | — | — | — | — |
| | PEG-20 stearate | 1.2 | 3.0 | 6.0 | — | — | — | — | — |
| | Polyoxyethylene | — | — | — | 1.2 | 6.0 | — | — | — |
| | hydrogenated | | | | | | | | |
| | castor oil | | | | | | | | |
| | Oleyl alcohol | — | — | — | — | — | 0.3 | 0.8 | 1.5 |
| | 1,3-butanediol | — | — | — | — | — | — | — | — |
| | Ethanol | 87 | 85 | 82 | 87 | 82 | 88 | 87 | 87 |
| Solution 2 | Water | 499 | 499 | 499 | 499 | 499 | 499 | 499 | 499 |
| | Total | 598 | 598 | 598 | 598 | 598 | 598 | 598 | 598 |

TABLE 9

| Quantitative | | | | | | | | | |
|---|---|---|---|---|---|---|---|---|---|
| measurement of | | | | | | | | | |
| various components | Production | Production | Production | Production | Production | Production | Production | Production | Production |
| contained in the lipid | Example | Example | Example | Example | Example | Example | Example | Example | Example |
| particle solutions | 10 | 11 | 12 | 13 | 14 | 15 | 16 | 17 | 18 |
| Hydrogenated | 1.2 | 1.2 | 1.2 | 1.2 | 1.2 | 1.2 | 1.2 | 1.2 | 1.2 |
| lecithin | | | | | | | | | |
| Cholesterol | 0.30 | 0.31 | 0.30 | 0.30 | 0.29 | 0.30 | 0.30 | 0.31 | 0.32 |
| Hydrogenated | 0.20 | 0.20 | 0.05 | 0.51 | 0.10 | 0.10 | 0.20 | 0.19 | 0.20 |
| lysolecithin | | | | | | | | | |
| N-stearoyl | 0.51 | 0.50 | 0.50 | 0.49 | 0.51 | 0.49 | 0.50 | 0.50 | 0.50 |
| dihydrosphingosine | | | | | | | | | |
| 1,3-butanediol | 7.4 | 8.8 | 7.2 | 7.2 | 7.3 | 8.8 | 7.2 | 0.0 | 0.0 |
| Ethanol | 7.2 | 5.9 | 7.4 | 7.1 | 7.3 | 5.9 | 7.4 | 14.6 | 14.1 |
| Water | 85 | 82 | 83 | 84 | 83 | 82 | 84 | 85 | 85 |

TABLE 10

| Quantitative | | | | | | | | |
|---|---|---|---|---|---|---|---|---|
| measurement of | | | | | | | | |
| various components | Production | Production | Production | Production | Production | Production | Production | Production |
| contained in the lipid | Example | Example | Example | Example | Example | Example | Example | Example |
| particle solutions | 19 | 20 | 21 | 22 | 23 | 24 | 25 | 26 |
| Hydrogenated | 1.2 | 1.2 | 1.2 | 1.2 | 1.2 | 1.2 | 1.2 | 1.2 |
| lecithin | | | | | | | | |
| Cholesterol | 0.31 | 0.30 | 0.30 | 0.29 | 0.29 | 0.30 | 0.31 | 0.29 |
| Hydrogenated | 0.00 | 0.00 | 0.00 | 0.00 | 0.00 | 0.00 | 0.00 | 0.00 |
| lysolecithin | | | | | | | | |
| N-stearoyl | 0.49 | 0.51 | 0.49 | 0.49 | 0.52 | 0.51 | 0.50 | 0.50 |
| dihydrosphingosine | | | | | | | | |
| 1,3-butanediol | 0.0 | 0.0 | 0.0 | 0.0 | 0.0 | 0.0 | 0.0 | 0.0 |
| Ethanol | 14.7 | 14.1 | 13.9 | 14.3 | 13.5 | 14.7 | 14.6 | 14.4 |
| Water | 84 | 84 | 86 | 82 | 85 | 83 | 83 | 85 |

### <Physical Properties and Stability of Lipid Particle Solution>

Various evaluations were performed on the lipid particle solutions obtained in Production Examples 10 to 26. The results are shown in Tables 11 and 12. All of the obtained lipid particle solutions immediately after the preparation were homogeneous and had no precipitation observed.

TABLE 11

| | | Production | Production | Production | Production | Production | Production | Production | Production | Production |
|---|---|---|---|---|---|---|---|---|---|---|
| Storage | Physical | Example | Example | Example | Example | Example | Example | Example | Example | Example |
| conditions | properties | 10 | 11 | 12 | 13 | 14 | 15 | 16 | 17 | 18 |
| Immediately | Average particle | 158 | 147 | 190 | 152 | 182 | 157 | 160 | 161 | 133 |
| after | size (nm) | | | | | | | | | |
| preparation | Polydispersity | 0.12 | 0.10 | 0.17 | 0.19 | 0.12 | 0.08 | 0.11 | 0.10 | 0.19 |
| | index | | | | | | | | | |
| | Coarse particle | 0 | 0 | 0 | 0 | 0 | 0 | 0 | 0 | 0 |
| | (>1 μm) content | | | | | | | | | |
| | (area %) | | | | | | | | | |
| | pH | 8.1 | 6.8 | 7.1 | 7.0 | 8.2 | 6.9 | 7.4 | 7.4 | 7.9 |
| | Zeta potential | −46 | — | — | — | — | — | — | −55 | −66 |
| | (mV) | | | | | | | | | |
| Stored at | Storage period | 3 | 1 | 1 | 1 | 3 | 1 | 1 | 1 | 1 |
| 4° C. | (months) | | | | | | | | | |
| | Average particle | 164 | 151 | 190 | 150 | 188 | 159 | 162 | 161 | 138 |
| | size (nm) | | | | | | | | | |
| | Polydispersity | 0.09 | 0.09 | 0.18 | 0.19 | 0.13 | 0.11 | 0.12 | 0.10 | 0.18 |
| | index | | | | | | | | | |
| | Coarse particle | 0 | 0 | 0 | 0 | 2 | 0 | 1 | 0 | 0 |
| | (>1 μm) content | | | | | | | | | |
| | (area %) | | | | | | | | | |
| | pH | 8.3 | 6.9 | 7.0 | 7.0 | 8.1 | 7.0 | 7.6 | 7.3 | 7.8 |
| | Appearance | No change | No change | No change | No change | No change | No change | No change | No change | No change |
| | | from the | from the | from the | from the | from the | from the | from the | from the | from the |
| | | state im- | state im- | state im- | state im- | state im- | state im- | state im- | state im- | state im- |
| | | mediately | mediately | mediately | mediately | mediately | mediately | mediately | mediately | mediately |
| | | after | after | after | after | after | after | after | after | after |
| | | prepara- | prepara- | prepara- | prepara- | prepara- | prepara- | prepara- | prepara- | prepara- |
| | | tion | tion | tion | tion | tion | tion | tion | tion | tion |
| Stored at | Storage period | 3 | 1 | 1 | 1 | 3 | 1 | 1 | 1 | 1 |
| 25° C. | (months) | | | | | | | | | |
| | Average particle | 162 | 150 | 191 | 149 | 194 | 157 | 164 | 165 | 140 |
| | size (nm) | | | | | | | | | |
| | Polydispersity | 0.11 | 0.11 | 0.17 | 0.19 | 0.10 | 0.08 | 0.10 | 0.10 | 0.17 |
| | index | | | | | | | | | |
| | Coarse particle | 0 | 0 | 0 | 0 | 0 | 0 | 0 | 0 | 0 |
| | (>1 μm) content | | | | | | | | | |
| | (area %) | | | | | | | | | |
| | pH | 8.2 | 6.8 | 7.0 | 6.9 | 8.1 | 7.0 | 8.2 | 7.6 | 7.7 |
| | Appearance | No change | No change | No change | No change | No change | No change | No change | No change | No change |
| | | from the | from the | from the | from the | from the | from the | from the | from the | from the |
| | | state im- | state im- | state im- | state im- | state im- | state im- | state im- | state im- | state im- |
| | | mediately | mediately | mediately | mediately | mediately | mediately | mediately | mediately | mediately |
| | | after | after | after | after | after | after | after | after | after |
| | | prepara- | prepara- | prepara- | prepara- | prepara- | prepara- | prepara- | prepara- | prepara- |
| | | tion | tion | tion | tion | tion | tion | tion | tion | tion |
| Stored at | Storage period | — | 1 | — | — | — | 1 | — | — | — |
| 40° C. | (months) | | | | | | | | | |
| | Average particle | — | 149 | — | — | — | 163 | — | — | — |
| | size (nm) | | | | | | | | | |
| | Polydispersity | — | 0.10 | — | — | — | 0.10 | — | — | — |
| | index | | | | | | | | | |
| | Coarse particle | — | 0 | — | — | — | 0 | — | — | — |
| | (>1 μm) content | | | | | | | | | |
| | (area %) | | | | | | | | | |
| | pH | — | 6.8 | — | — | — | 6.8 | — | — | — |
| | Appearance | No change | No change | — | — | — | No change | — | — | — |
| | | from the | from the | | | | from the | | | |
| | | state im- | state im- | | | | state im- | | | |
| | | mediately | mediately | | | | mediately | | | |
| | | after | after | | | | after | | | |
| | | preparation | preparation | | | | preparation | | | |
| Stored at | Storage period | 1 | 1 | 1 | 1 | 1 | 1 | 1 | 1 | 1 |
| 50° C. | (months) | | | | | | | | | |
| | Average particle | 172 | 152 | 198 | 150 | 210 | 162 | 163 | 183 | 135 |
| | size (nm) | | | | | | | | | |
| | Polydispersity | 0.11 | 0.10 | 0.16 | 0.19 | 0.11 | 0.09 | 0.11 | 0.11 | 0.19 |
| | index | | | | | | | | | |
| | Coarse particle | 2 | 0 | 0 | 0 | 0 | 0 | 0 | 0 | 0 |
| | (>1 μm) content | | | | | | | | | |
| | (area %) | | | | | | | | | |
| | pH | 7.9 | 6.6 | 6.7 | 6.6 | 8.2 | 6.5 | 7.8 | 7.9 | 7.7 |
| | Appearance | No change | No change | No change | No change | No change | No change | No change | No change | No change |
| | | from the | from the | from the | from the | from the | from the | from the | from the | from the |
| | | state im- | state im- | state im- | state im- | state im- | state im- | state im- | state im- | state im- |
| | | mediately | mediately | mediately | mediately | mediately | mediately | mediately | mediately | mediately |
| | | after | after | after | after | after | after | after | after | after |
| | | prepara- | prepara- | prepara- | prepara- | prepara- | prepara- | prepara- | prepara- | prepara- |
| | | tion | tion | tion | tion | tion | tion | tion | tion | tion |

TABLE 12

| | | Production | Production | Production | Production | Production | Production | Production | Production |
|---|---|---|---|---|---|---|---|---|---|
| Storage | Physical | Example | Example | Example | Example | Example | Example | Example | Example |
| conditions | properties | 19 | 20 | 21 | 22 | 23 | 24 | 25 | 26 |
| Immediately | Average particle | 205 | 214 | 222 | 216 | 200 | 120 | 127 | 144 |
| after | size (nm) | | | | | | | | |
| preparation | Polydispersity | 0.17 | 0.18 | 0.20 | 0.14 | 0.21 | 0.12 | 0.15 | 0.16 |
| | index | | | | | | | | |
| | Coarse particle | — | — | — | — | — | — | — | — |
| | (>1 μm) content | | | | | | | | |
| | (area %) | | | | | | | | |
| | pH | — | — | — | — | — | — | — | — |
| | Zeta potential | — | — | — | — | — | — | — | — |
| | (mv) | | | | | | | | |
| Stored at | Storage period | 0.25 | 0.25 | 0.25 | 0.25 | 0.25 | 0.25 | 0.25 | 0.25 |
| 4° C. | (months) | | | | | | | | |
| | Average particle | 216 | 214 | 1203 | 214 | 234 | 120 | 127 | 144 |
| | size (nm) | | | | | | | | |
| | Polydispersity | 0.19 | 0.18 | 0.42 | 0.15 | 0.22 | 0.12 | 0.15 | 0.16 |
| | index | | | | | | | | |
| | Coarse particle | — | — | — | — | — | — | — | — |
| | (>1 μm) content | | | | | | | | |
| | (area %) | | | | | | | | |
| | pH | — | — | — | — | — | — | — | — |
| | Appearance | With | With | With | With | With | With | With | With |
| | | precipitation | precipitation | precipitation | precipitation | precipitation | precipitation | precipitation | precipitation |

From the results of Tables 11 and 12, it was found that each of the lipid particle solutions of Production Examples 10 to 18 of the present invention, even after the storage at various temperatures, maintained the average particle size, polydispersity index, content of coarse particles, pH, and appearance immediately after the preparation, was in a homogeneous state without precipitation or deposition, and has excellent storage stability.

### <Blended Composition>

Formulated products containing the lipid particle solution obtained in Production Example 10 or Production Example 14 (blended compositions 5 to 7) and formulated products using formulations containing no lipid particle solution (blended compositions 8 and 9) were prepared with the ingredients shown in Table 6 and the formulations (unit: part) shown in Table 13. Immediately after the preparation, any blended composition was a uniform product without precipitation or deposition and was in a state worthy of evaluation of storage stability and use feeling.

TABLE 13

| | Blended | Blended | Blended | Blended | Blended |
|---|---|---|---|---|---|
| | composition | composition | composition | composition | composition |
| Component (ingredient) | 5 | 6 | 7 | 8 | 9 |
| Ultrapure water | 71.525 | 71.525 | 69.305 | 88.305 | 86.085 |
| (Purified water (obtained by | | | | | |
| “Milli-Q (registered trademark)” | | | | | |
| manufactured by Merck) | | | | | |
| 1,3-butanediol | 6.0 | 6.0 | 8.0 | 7.7 | 9.7 |
| Glycerin | 2.0 | 2.0 | 2.0 | 2.0 | 2.0 |
| Xanthan gum | 0.05 | 0.05 | — | 0.05 | — |
| Phenoxyethanol | 0.30 | 0.30 | 0.30 | 0.30 | 0.30 |
| Carbomer | 0.10 | 0.10 | — | 0.10 | — |
| Sodium hydroxide | 0.025 | 0.025 | — | 0.025 | — |
| Citrcitric acid | — | — | 0.04 | — | 0.04 |
| Sodium citrate | — | — | 0.36 | — | 0.36 |
| Ethanol | — | — | — | 1.5 | 1.5 |
| Lipid particle solution of | 20 | — | 20 | — | — |
| Production Example 10 | | | | | |
| Lipid particle solution of | — | 20 | — | — | — |
| Production Example 14 | | | | | |
| Total | 100 | 100 | 100 | 100 | 100 |

### <Physical Properties and Stability of Blended Composition>

Various evaluations were performed on the blended compositions 5 to 7 obtained. The results are shown in Table 14.

TABLE 14

| | | Blended | Blended | Blended |
|---|---|---|---|---|
| Storage | Physical | composition | composition | composition |
| conditions | properties | 5 | 6 | 7 |
| Immediately | Average particle | 151 | 161 | 153 |
| after | size (nm) | | | |
| preparation | Polydispersity | 0.15 | 0.12 | 0.09 |
| | index | | | |
| | Coarse particle | 1 | 0 | 0 |
| | (>1 μm) content | | | |
| | (area %) | | | |
| | pH | 6.9 | 7.0 | 6.4 |
| | Appearance | ○ | ○ | ○ |
| Stored | Storage period | 1 | 1 | 1 |
| at 4° C. | (months) | | | |
| | Average particle | 158 | 168 | 172 |
| | size (nm) | | | |
| | Polydispersity | 0.13 | 0.11 | 0.13 |
| | index | | | |
| | Coarse particle | 0 | 0 | 0 |
| | (>1 μm) content | | | |
| | (area %) | | | |
| | pH | 7.2 | 7.1 | 6.5 |
| | Appearance | ○ | ○ | ○ |
| Stored | Storage period | 1 | 1 | 1 |
| at 50° C. | (months) | | | |
| | Average particle | 155 | 169 | 158 |
| | size (nm) | | | |
| | Polydispersity | 0.16 | 0.12 | 0.06 |
| | index | | | |
| | Coarse particle | 0 | 5 | 0 |
| | (>1 μm) content | | | |
| | (area %) | | | |
| | pH | 6.7 | 6.7 | 6.7 |
| | Appearance | ○ | ○ | ○ |

In the appearance evaluation shown in Table 14, an appearance in a uniform state without precipitation or deposition was judged as ∘.

From the results in Table 14, it was found that the blended compositions 5 to 7 containing the lipid particle solutions of the present invention are excellent in long-term stability at low and high temperatures. In addition, from the results of the blended composition 7, it was found that even the composition containing no water-soluble polymer is excellent in storage stability.

The lipid particle solutions of Production Examples 10 and 14 shown in Table 7, and the blended compositions 5 to 9 shown in Table 13 were evaluated for the use feeling. The results are shown in Table 15.

TABLE 15

| | | Production | Production | Blended | Blended | Blended | Blended | Blended |
|---|---|---|---|---|---|---|---|---|
| | | Example 10 | Example 14 | composition 5 | composition 6 | composition 7 | composition 8 | composition 9 |
| Use feeling | Smoothness | ∘ | ∘ | ∘ | ∘ | ∘ | x | x |
| evaluation | Skin | ∘ | ∘ | ∘ | ∘ | ∘ | x | x |
| (during | penetration | | | | | | | |
| application) | Rich texture | ∘ | ∘ | ∘ | ∘ | ∘ | x | x |
| | Skin freshness | Yes | Yes | Yes | Yes | Yes | Yes | Yes |
| | sensation | | | | | | | |
| Use feeling | Moisture | ∘ | ∘ | ∘ | ∘ | ∘ | x | x |
| evaluation | retention | | | | | | | |
| (after | Persistence of | ∘ | ∘ | ∘ | ∘ | ∘ | x | x |
| application) | skin resilience | | | | | | | |

### <Electron Microscope Observation>

After pretreatment by an ice embedding method, a lipid particle of Production Example 10 was observed with a transmission electron microscope. The obtained image is shown in FIG. 7.

From the results of FIG. 7, it was found that the lipid particle contained in the lipid particle solution of Production Example 10 is spherical.

### Production Examples 27 to 29: Addition of Additive to Lipid Particle Solution

To 10 mL of the lipid particle solution obtained in Production Example 2 was added an additive (storage stability improving agent) at various concentrations, and each of the resulting solutions was adjusted in pH and placed in a glass container. After the solution was stored at 4° C., 25° C., or 50° C. for one month, the changes in the average particle size and pH were observed. The results are shown in Table 16.

TABLE 16

| | | Production | Production | Production |
|---|---|---|---|---|
| | | Example 27 | Example 28 | Example 29 |
| Additive | | Triethanolamine | Calcium hydroxide | Arginine |
| Product name | | 2,2′,2″- | Calcium hydroxide | L(+)-arginine |
| | | Nitrilotriethanol | (Guaranteed | |
| | | (Guaranteed | reagent) | |
| | | reagent) | | |
| Manufacturer | | Fujifilm Wako Pure Chemical Corporation | | |
| Addition amount (ppm) | | 875 | 32 | 750 |
| Storage | Physical | | | |
| conditions | properties | | | |
| Immediately | Average particle | 236 | 247 | 238 |
| after addition | size (nm) | | | |
| | Polydispersity | 0.22 | 0.23 | 0.24 |
| | index | | | |
| | pH | 7.2 | 6.8 | 7.1 |
| | Appearance | ○ | ○ | ○ |
| Stored at | Storage period | 1 | 1 | 1 |
| 4° C. | (months) | | | |
| | Average particle | 248 | 248 | 236 |
| | size (nm) | | | |
| | Polydispersity | 0.21 | 0.23 | 0.22 |
| | index | | | |
| | pH | 7.2 | 7.0 | 6.8 |
| | Appearance | ○ | ○ | ○ |
| Stored at | Storage period | 1 | 1 | 1 |
| 25° C. | (months) | | | |
| | Average particle | 250 | 241 | 253 |
| | size (nm) | | | |
| | Polydispersity | 0.23 | 0.22 | 0.22 |
| | index | | | |
| | pH | 7.2 | 6.9 | 6.8 |
| | Appearance | ○ | ○ | ○ |
| Stored at | Storage period | 1 | 1 | 1 |
| 50° C. | (months) | | | |
| | Average particle | 256 | 261 | 249 |
| | size (nm) | | | |
| | Polydispersity | 0.24 | 0.23 | 0.22 |
| | index | | | |
| | pH | 6.9 | 6.5 | 6.7 |
| | Appearance | ○ | ○ | ○ |

In the appearance evaluation shown in Table 1.6, an appearance in a uniform state without precipitation or deposition was judged as ∘.

From the results in Table 16, it was found that each of the lipid particle solutions prepared in Production Examples 27 to 29 after storage at various temperatures maintained the initial states of appearance color, appearance condition, average particle size, and pH when initiating the storage. From this, it was found that the lipid particle solutions prepared in Production Examples 27 to 29 had dramatically improved storage stability compared with the lipid particle solution prepared in Production Example 2.

In the use feeling evaluation of the lipid particle solutions prepared in Production Examples 27 to 29, the judgments on stickiness were ∘, and it was confirmed that no stickiness was felt. In addition, in the evaluations of smoothness, skin penetration, and rich texture, the judgments on all the items were ∘.

### Production Example 30: Preparation of Lipid Particle Solution for Skin Barrier Properties Evaluation Test Using Three-Dimensional Skin Model

Using the ingredients shown in Table 17, a lipid particle solution (cosmetic composition 30 of the present disclosure) was prepared in the same manner as in Production Example 10 except that the formulation was changed to the formulation (unit: part) as shown in Table 17.

TABLE 17

| | | | Production |
|---|---|---|---|
| | Component | Product name | Example 30 |
| Solution 1 | Hydrogenated lecithin | Phospholipon90H | 2.6 |
| | (hydrogenated soybean | | |
| | phosphatidylcholine) | | |
| | Cholesterol | Cholesterol | 0.7 |
| | | NF-PW-(JP) | |
| | Hydrogenated lysolecithin | SLP-LPC70H | 0.4 |
| | (hydrogenated soybean | | |
| | lysophospholipid) | | |
| | N-stearoyl | Ceramide TIC-001 | 1.1 |
| | dihydrosphingosine | | |
| | (ceramide NG) | | |
| | 1,3-butanediol | 1,3-butanediol | 42 |
| | Ethanol | Ethanol | 42 |
| Solution 2 | Water | Tris buffer solution | 444 |
| Total | | | 533 |

The Tris buffer solution shown in Table 17 is a pH 7.5 solution obtained by dissolving 0.1 M sodium chloride in a 20 mM tris(hydroxymethyl)aminomethane hydrochloride aqueous solution (hereinafter referred to as Tris buffer solution). The lipid particle solution obtained in Preparation Example 30 had an average particle size of 210 nm and a polydispersity index of 0.19.

### <Skin Barrier Properties Evaluation Test Using Three-Dimensional Skin Model>

A skin barrier properties evaluation test was performed using the lipid particle solution prepared in Production Example 30 as a sample solution. T-Skin (reproduced human skin full-thickness model) commercialized by Nikoderm Research Inc. (hereinafter referred to as Nikoderm) was used as a three-dimensional skin model. The three-dimensional skin model was cut out from the bottom of a transwell of T-Skin. As shown in FIG. 2, a three-dimensional skin model 4 was attached to a vertical Franz diffusion cell 7 (effective diffusion area: 1.13 cm²) with the dermis side of the three-dimensional skin model 4 facing a receiver solution 1 side, and a sample liquid 5 was applied to the epidermal layer side of the three-dimensional skin model 4. In FIG. 2, a constant temperature circulating water inlet 2 and a constant temperature circulating water outlet 3 were connected to a constant temperature bath, and constant temperature water of 37° C. was flowed from the constant temperature circulating water inlet 2 toward the constant temperature circulating water outlet 3 and circulated around the outer periphery of the vertical Franz diffusion cell 7. The application volume of the sample solution was 1 mL per 1 cm² of effective diffusion area. 3.7 mL of Tris acid buffer (pH 7.5) was used as the receiver solution. During the test, the receiver solution 1 was stirred by rotating a stirrer 6 placed in the receiver solution 1 with a magnetic stirrer 8. The skin barrier properties evaluation test was performed in a laboratory at room temperature of 25° C. and humidity of 60%. After 120 minutes of the application of the sample solution, the applied sample solution was removed, and a Tris buffer solution (pH 7.5) containing 10 mg/ml of caffeine (manufactured by Fujifilm Wako Pure Chemical Corporation) was applied. The receiver solution 1 was sampled at 0.25 mL 60 minutes after application of the caffeine solution.

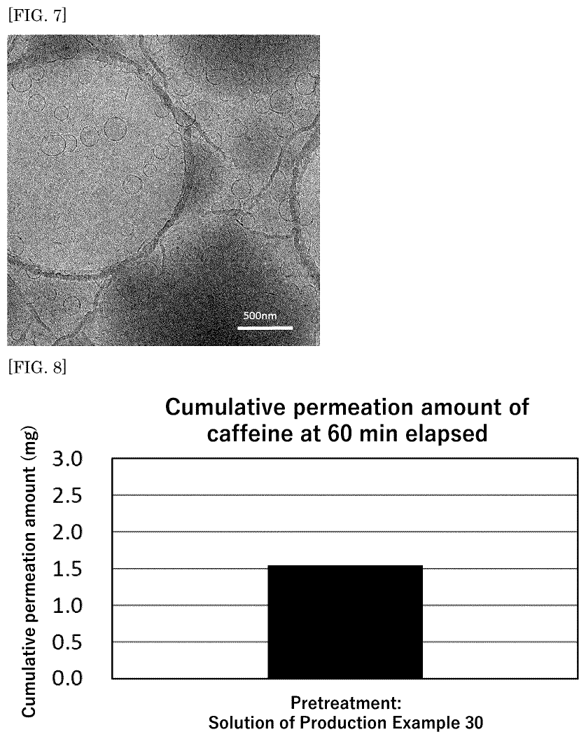

The obtained receiver solution 1 was diluted 3 to 10-fold with a Tris buffer solution, and the amount of caffeine in the solution was quantified by liquid chromatography. The result is shown in FIG. 8.

1. A cosmetic composition or cosmetic quasi-drug comprising:
lipid particles containing at least a phospholipid;
a polyhydric alcohol; and
water,
wherein a content of a trihydric or higher alcohol in the polyhydric alcohol is 0 to 20% by mass relative to 100% by mass of the polyhydric alcohol.

2. The cosmetic composition or cosmetic quasi-drug according to claim 1, wherein the lipid particles have an average particle size of 10 nm or more.

3. The cosmetic composition or cosmetic quasi-drug according to claim 1, wherein an absolute value of a zeta potential of a surface of the lipid particles is 5 mV or more.

4. The cosmetic composition or cosmetic quasi-drug according to claim 1, wherein a rate of change in average particle size of the lipid particles after storage at 25° C. for 1 month is ±20% or less.

5. The cosmetic composition or cosmetic quasi-drug according to claim 1 which is produced using a microfluidic device.
